(19) United States
(12) Patent Application Publication
(10) Pub. No.: US 2021/0030651 A1
(43) Pub. Date: Feb. 4, 2021

(54) HAIR TREATMENT COMPOSITIONS COMPRISING REDUCING AGENTS

(71) Applicant: L'OREAL, Paris (FR)

(72) Inventors: Marina Tavares GREGOLIN, Rio De Janeiro-RJ (BR); Pedro Henrique CARVALHO, Rio De Janeiro-RJ (BR); Manon CHAUMONTET, Clichy (FR); Sébastien VOISIN, Saint-Ouen (FR); Chantal JOUY, Saint-Ouen (FR)

(21) Appl. No.: 17/046,072

(22) PCT Filed: Apr. 9, 2018
(86) PCT No.: PCT/BR2018/050095

§ 371 Date: Oct. 8, 2020

(51) Int. Cl. A61K 8/46; A61K 8/362; A61K 8/41; A61K 8/34; A61K 8/42; A61K 8/55; A61K 8/891; A61K 8/898; A61Q 5/04

(52) U.S. Cl. CPC A61K 8/46; A61K 8/362; A61K 8/416; A61K 8/342; A61K 8/42; A61K 2800/596; A61K 8/891; A61K 8/898; A61Q 5/04; A61K 2800/30; A61K 2800/48; A61K 8/55

(57) ABSTRACT

The present disclosure relates to hair treatment compositions comprising: (a) at least one reducing agent selected from thiol-based compounds, non-thiol-based compounds, and mixtures thereof; (b) at least one non-polymeric and non-thiol, mono-, di-, and/or tri-carboxylic acid, and/or a salt thereof; (c) at least one alkaline agent; and (d) optionally, at least one cationic surfactant; wherein the pH of the composition is from about 2 to less than 7. The disclosure also relates to methods for imparting hair care benefits to the hair such as straightening effects, volume reduction, frizz control, manageability, cosmeticity and smooth feel, in addition to conditioning benefits.

### FIELD OF THE DISCLOSURE

The present disclosure relates to hair treatment compositions comprising reducing agents, such as thiol-based compounds, and to methods for providing cosmetic and caring benefits to hair using the hair treatment compositions.

### BACKGROUND OF THE DISCLOSURE

The quality and condition of hair is generally adversely affected by the action of external agents such as sunlight, wind, pollution, humidity, and chemicals in swimming pools, and also by mechanical or chemical treatments, such as brushing, combing, shampooing, dyeing, bleaching, permanent-waving and/or relaxing. Hair is thus damaged by these various factors and may over time become dry, coarse or dull, especially in fragile areas, and more particularly at the ends leading to split ends. Under such circumstances, hair can also become less manageable, more frizzy and less disciplined, and more difficult to style or shape.

Hair care products are used to combat these drawbacks. Conventional cleansing compositions such as shampoos, for example, which contain surfactants such as anionic, nonionic and/or amphoteric type surfactants, can be employed to remove the diverse types of soils typically present on hair. These cleansing compositions, while providing good cleansing power, may result in a less conditioned or rough feel to the hair due to, for example, the gradual removal of the natural or applied fats, lipids, or proteins contained in or at the surface of the hair. In addition, washing hair with water alone can also cause the hair to become tangled, less smooth and dry.

Hair conditioner or hair masque (mask) products can be used on hair after it has been shampooed or rinsed in order to confer hair caring properties to hair. Traditional conditioners and masques provide conditioning, smoothing, softening and visual sleekness to hair and are generally rinse-off products.

Consumers also seek hair caring regimens and products that can provide control, or even eliminate frizziness, and control or reduce the volume and the apparent mass of hair on the head as well as provide additional benefits of changing the appearance, shape or configuration of hair. In addition, consumers desire that such products impart longer-lasting or continuous benefits to hair over a period of time. Thus, it is desirable for manufacturers of hair products to formulate not only rinse-off shampoos and conditioners but also products that do not involve immediate rinsing or that provide longer term treatments such as those provided by leave-on hair products.

One type of product that can shape or configure hair are hair styling products, for example, leave-in gels and creams which employ ingredients such as film forming polymers that impart desirable styling or shaping hold while on the hair. However, some ingredients can be easily removed from the hair, for example by rinsing or washing. Thus, any cosmetic benefits to the hair from such products are generally diminished or removed once the hair is rinsed or washed.

Another type of composition that can be applied onto hair in order to change its shape and make it more manageable is an alkaline composition that is found in commercial hair relaxers or hair straighteners. These products can confer longer lasting shape or style to the hair. Alkaline hair relaxing/straightening involves hydrolyzing the keratin of the hair with various alkaline agents, such as inorganic hydroxides, for instance sodium hydroxide, or organic hydroxides, such as guanidine hydroxide, or organic amines. Hair relaxing/straightening products that employ sodium hydroxide or potassium hydroxide are also called lye-based products and products that use other alkaline agents such as lithium hydroxide, calcium hydroxide, organic hydroxides and other non-hydroxide compounds, for example, organic amines, generally fall under the category of no-lye products. Still, it is desirable to find alternatives to the alkaline lye- and no-lye-based products and process described above which can damage the hair by weakening and/or causing dryness of the hair fibers.

Thus, it is highly desirable to have hair care regimens and products that, for example, relax or straighten curly or kinky hair, including wavy hair, or reduce/loosen curls while making the hair more manageable, untangled, more easily styled or shaped, and disciplined. At the same time, it is also highly desirable that such regimens and products confer cosmetic effects that are long-lasting.

It is an object of embodiments of the disclosure to provide a hair treatment composition with ingredients that can effectively condition the hair and at the same time, impart a light weight feel, good styling/shaping or straightening benefits as well as enhanced hair caring and manageability properties and other cosmetic properties such as volume control/reduction, frizz control/reduction, discipline (less fly-aways), and regularity of shape.

However, the discovery of new compositions and processes for treating hair that have enhanced efficacy but impart less or minimal damage to hair, may pose challenges to manufacturers and formulators because the incorporation of new ingredients into the compositions may negatively impacting their performance, cosmetic attributes, texture, and formulation stability. In addition, the acidity/alkalinity and/or pH is an important consideration for these products. New processes for treating and changing the shape of hair may also impact the performance of the compositions, processing times and quality of use. Thus, manufacturers of such products continuously test the use of new raw materials and ingredients or new product forms and seek to re-formulate and create new products with the desired qualities, while still remaining stable and safe to use.

### SUMMARY OF THE DISCLOSURE

The present disclosure is directed to hair treatment compositions comprising:
at least one reducing agent selected from thiol-based compounds, non-thiol-based compounds, and a mixture thereof;
at least one non-polymeric and non-thiol, mono-, di-, and/or tri-carboxylic acid, and/or a salt thereof, or a mixture thereof;
at least one alkaline agent;
optionally, at least one cationic surfactant; and
water;

wherein the pH of the composition is from about 2 to less than 7.

In addition to the reducing agent(s), the non-polymeric and non-thiol, mono-, di-, and/or tri-carboxylic acid(s) and/or salt(s) thereof, the alkaline agent(s), the optional cationic surfactant(s), and water, in some embodiments the hair treatment compositions may additionally include at least at least one nonionic surfactant, at least one thickening agent, at least one silicone compound, and/or at least one water-soluble solvent.

The present disclosure is also directed to methods for treating the hair involving the application of the above-described composition onto hair, and optionally heating the hair.

### BRIEF DESCRIPTION OF THE DRAWINGS

Implementations of the present disclosure will now be described, by way of example only, with reference to the attached FIGURES, wherein:

FIG. 1 compares hair swatches treated with comparative formulations to hair swatches treated with inventive compositions.

It should be understood that the various aspects are not limited to the arrangements and instrumentality shown in the drawings.

### DETAILED DESCRIPTION OF THE DISCLOSURE

The present hair treatment compositions effectively condition the hair and provide manageability properties to hair, including one or more of straightening effects; frizz control; volume reduction or volume control; light weight feel on the hair, styling or shaping effects; curling effects; relaxing effects or reduction/loosening of curls; improvement or retention of curl definition; humidity resistance; cosmeticity to the feel; smooth feel; natural feel; less or reduced rough ends; and/or improvement of the appearance of hair.

“At least one” as used herein is synonymous with “one or more” and includes individual components as well as mixtures/combinations.

“Conditioning” as used herein means imparting to one or more hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the hair treatment compositions of the present disclosure onto the surface of hair. The term ‘treat” (and its grammatical variations) as used herein also refers to contacting hair with the hair treatment compositions of the present disclosure.

A “rinse-off” product refers to a composition such as a hair care composition that is rinsed and/or washed with water either after or during the application of the composition onto the keratinous substrate, and before drying and/or styling said keratinous substrate. At least a portion of the composition is removed from the keratinous substrate during the rinsing and/or washing.

A “leave-on” (also called leave-in) product refers to a hair treatment composition such as a hair treatment composition that is applied to a keratinous substrate such as hair and not further subjected to a rinsing and/or washing step for at least 1 hour or for a period of time ranging from 1 hour up to 72 hours such as from 4 hours up to 48 hours or from 8 hours up to 36 hours or from 8 hours up to 24 hours.

The term “stable” as used herein means that the composition does not exhibit phase separation and/or crystallization.

The term “volatile”, as used herein, means having a flash point of less than about 100° C.

The term “non-volatile”, as used herein, means having a flash point of greater than about 100° C.

The term “reducing agent” as used herein, means an agent capable of reducing the disulfide bonds of the hair.

The term “active material” as used herein with respect to the percent amount of an ingredient or raw material, refers to 100% activity of the ingredient or raw material.

The term “substituted,” as used herein, means comprising one or more substituents. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

The hair treatment compositions and methods of the present disclosure can comprise, consist of, or consist essentially of the essential elements and limitations of the disclosure described herein, as well as any additional or optional ingredients, components, or limitations described herein or otherwise useful.

Other than in the operating examples, or where otherwise indicated, all numbers expressing quantities of ingredients and/or reaction conditions are to be understood as being modified in all instances by the term “about,” meaning within +/−5% of the indicated number.

All percentages, parts and ratios herein are based upon the total weight of the compositions of the present disclosure, unless otherwise indicated.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from A, B, C, D, E, F, and a mixture of any two or more of A, B, C, D, E, and F.”

Likewise, the term “a salt thereof” also relates to “salts thereof.” Thus, where the disclosure refers to “an element selected from A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

The present disclosure relates to a hair treatment composition comprising:
at least one reducing agent selected from thiol-based compounds, non-thiol-based compounds, and a mixture thereof;
at least one non-polymeric and non-thiol, mono-, di-, and/or tri-carboxylic acid, and/or a salt thereof, or a mixture thereof;
at least one alkaline agent;
optionally, at least one cationic surfactant; and
water;

wherein the pH of the composition is from about 2 to less than 7.

A particularly useful and preferred thiol-based compound is thiolactic acid.

The total amount of the at least one reducing agent in the hair treatment compositions may vary but is typically about 0.1 to about 20 wt. %, preferably about 0.5 to about 16 wt. %, more preferably about 1 to about 14 wt. %, even more preferably about 2 to about 12 wt. %, and even more preferably about 4 to about 10 wt. %, and even more preferably about 6 to about 10 wt. %, based on the total weight of the hair treatment composition.

The hair treatment compositions include at least one non-polymeric and non-thiol, mono-, di-, and/or tri-carboxylic acid, a salt thereof, or a mixture thereof. Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof. In some instances, lactic acid and/or a salt thereof is a preferred monocarboxylic acid.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof. In some instances, malic acid, glutaric acid, a salt thereof, or a mixture thereof, are preferable dicarboxylic acids.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof. In some instances, citric acid and/or a salt thereof, are preferred tricarboxylic acids.

In some embodiments, the hair treatment compositions include at least two, three, or four non-polymeric and non-thiol, mono-, di-, and/or tricarboxylic acids, and/or salts thereof.

The hair treatment compositions of the present disclosure include at least one alkaline agent. Non-limiting examples of useful alkaline agents include organic amines, ammonium, alkali metal hydroxides, alkali earth metal hydroxides, alkali metal carbonates, alkali metal phosphates, alkali metal citrates, amino acids (for example, L-lysine, L-arginine, etc.), and mixtures thereof, and preferably those selected from aminomethyl propanol, sodium hydroxide, potassium hydroxide, lithium hydroxide, aminomethyl propanediol, triisopropanol amine, dimethylstearylamine, dimethyl/tallowamine, lysine, ornithine, arginine, monoethanolamine, triethanolamine, calcium hydroxide, calcium bicarbonate, and mixtures thereof. In one embodiment, the alkaline agent is a diamine, for example, a diamine such as tetrahydroxyethyl ethylenediamine, tetrahydroxypropyl ethylenediamine, tetrahydroxypropyl ethylenediamine dioleate, or a mixture thereof. In one embodiment, a preferred alkaline agent is an alkali metal hydroxide and/or an alkali earth metal hydroxide, preferably sodium hydroxide, potassium hydroxide, lithium hydroxide, or a mixture thereof. In one embodiment, particularly preferred alkaline agents are organic amines, especially alkanolamines, for example an alkanolamine selected from monoethanolamine, diethanolamine, triethanolamine, tris(hydroxymethyl)aminomethane, tetra hydroxy propyl ethylenediamine, and a mixture thereof; and most preferably triethanolamine. In one embodiment, the alkaline agent is selected from sodium hydroxide, potassium hydroxide, triethanolamine, aminomethyl propanol, and a mixture thereof.

The total amount of the at least one alkaline agent in the hair treatment composition can vary but is typically about 0.01 to about 15 wt. %, preferably about 0.1 to about 12 wt. %, more preferably about 0.2 to about 10 wt. %, and even more preferably about 0.5 to about 8 wt. %, based on the total weight of the hair treatment composition.

Non-limiting examples of cationic surfactants that may be included in the hair treatment compositions include polyoxyalkenylated primary, secondary, tertiary fatty amine salts, quaternary ammonium compounds and their salts, and mixtures thereof, and preferably quaternary ammonium compounds and their salts, and more preferably those selected from cetrimonium chloride, behentrimonium chloride, dipalmitoylethylhydroxyethylmonium methosulfate, behentrimonium methosulfate, and mixtures thereof. A more exhaustive list of useful cationic surfactants that may be included is provided later, under the heading “Cationic Surfactant.”

The total amount of the at least one cationic surfactant in the hair treatment compositions, if present, may vary but is typically about 0.1 to about 15 wt. %, preferably, 0.5 to about 10 wt. %, and more preferably about 1 to about 6 wt. %, based on the total weight of the hair treatment composition.

In certain embodiments, the hair treatment compositions of the instant disclosure include at least one nonionic surfactant. Non-limiting examples of nonionic surfactants include fatty alcohols, alkoxylated fatty alcohols, alkyl(ether)phosphates, alkylpolyglucosides, fatty acid alkanolamides, and mixtures thereof. In one embodiment, the at least one nonionic surfactant is selected from linear, branched saturated/unsaturated fatty alcohols comprising from 6 to 60 carbon atoms and preferably from cetyl alcohol, stearyl alcohol, cetearyl alcohol (mixture of cetyl alcohol and stearyl alcohol), octyldodecanol, isostearyl alcohol, 2-hexyl decanol, palmityl alcohol, myristyl alcohol, stearyl alcohol, lauryl alcohol, oleic alcohol (or oleyl), linoleyl alcohol (or linoley-ether), linolenic alcohol (or linolenyl) and undecylenic alcohol, and mixtures thereof, and more preferably from cetyl alcohol, stearyl alcohol, and cetearyl alcohol, steareth-20, PPG-5-Ceteth-10 phosphate, Oleth-3 phosphate, Oleth-10 phosphate, Ceteth-10 phosphate, a mixture of Ceteth-10 phosphate and Dicetyl phosphate, Dicetyl phosphate, Cetyl phosphate, Stearyl phosphate, laureth-7, laureth-9, trideceth-10, trideceth-12, C12-13 pareth-3, C12-13 pareth-23, C11-15 pareth-7, PPG-5 ceteth-20, PEG-55 Propylene Glycol Oleate, glycereth-26, decyl glucoside, cetearyl glucoside, decyl lauryl glucoside, stearyl glucoside, coco-glucoside, cocamide MIPA, and mixtures thereof. A more exhaustive list of useful nonionic surfactants that may be included is provided later, under the heading “Nonionic Surfactant.”

In one embodiment of the disclosure, the total amount of the at least one nonionic surfactant may vary but is typically about 0.1 to about 15 wt. %, preferably about 0.5 to about 12 wt. %, more preferably about 1 to about 10 wt. %, and even more preferably about 2 to about 7 wt. %, based on the total weight of the hair treatment composition.

In certain embodiments, the hair treatment compositions of the present disclosure include at least one thickening agent. Non-limiting examples of useful thickening agents include cellulose polymers, gums, modified or unmodified carboxyvinyl polymers, polyacrylamides, copolymers of acrylic acid and of acrylamide, sodium salts of polyhydroxycarboxylic acids, optionally crosslinked and/or neutralized 2-acrylamido-2-methylpropanesulphonic acid polymers and copolymers, polyacrylic acid/alkyl acrylate, glucans, modified or unmodified starches, silicas, and mixtures thereof.

In a preferred embodiment, the at least one thickening agent comprises a gum, especially those derived from natural sources. Non-limiting examples of useful gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof. Particularly preferred gums include xanthan gum, guar gum, biosaccharide gum, cellulose, acacia gum, seneca gum, sclerotium gum, agarose, gellan gum, and mixtures thereof. In some instances a combination of xanthan gum and sclerotium gum may be particularly useful. A more exhaustive list of useful thickening agents that may be included is provided later, under the heading “Thickening Agent.”

The total amount of the at least one thickening agent in the hair treatment compositions can vary but is typically about 0.01 to about 10, preferably about 0.05 to about 8, more preferably about 0.1 to about 6 wt. %, and even more preferably about 0.2 to about 4 wt. %, based on the total weight of the hair treatment composition.

In some embodiments of the instant disclosure, the hair treatment compositions include at least one silicone compound. Non-limiting examples of useful silicone compounds include dimethicone; aminosilicones such as amodimethicone, trideceth-9 PG amodimethicone, PEG-40/PPG-8 methylaminopropyl/hydroxypropyl dimethicone copolymer; dimethicone copolyols selected from oxypropylenated and/or oxyethylenated polydimethyl(methyl)siloxane, oxypropylenated and/or oxyethylenated polymethyl (C8-C22) alkyl dimethyl methyl siloxane, and mixtures thereof. Particularly preferred silicone compounds include dimethicone and/or amodimethicone. A more exhaustive list of useful silicone compounds that may be included is provided later, under the heading “Silicone Compound.”

The total amount of the at least one silicone compound in the hair treatment compositions may vary but is typically about 0.1 to about 15 wt. %, preferably about 0.5 to about 10 wt. %, more preferably about 1 to about 5 wt. %, based on the total weight of the hair treatment composition.

In one embodiment of the present disclosure, the pH of the hair treatment compositions may range from about 2.5 to about 6.5, or from about 3 to about 6, or from about 3 to about 5.2, such as from about 3 to about 5, or preferably from about 3 to about 4.8, or more preferably from about 3 to about 4.5, or even more preferably from about 3 to about 4, including all ranges and sub ranges therebetween.

In one embodiment of the present disclosure, the hair treatment composition is a leave-on composition. Furthermore, in some embodiments, the hair treatment compositions may be in the form of a gel, a paste, a lotion, a cream, or a spray.

In one embodiment of the present disclosure, the hair treatment composition includes:
about 0.1 to about 20 wt. %, preferably about 0.5 to about 16 wt. %, more preferably about 1 to about 14 wt. % of at least one thiol-based compound selected from thiolactic acid, an ester thereof, a salt thereof, and a mixture thereof, preferably thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of the at least one non-polymeric and non-thiol, mono-, di-, and/or tricarboxylic acid, a salt thereof, or a mixture thereof, preferably, lactic acid, malic acid, glutaric acid, citric acid, a salt thereof, or a mixture thereof, based on the total weight of the hair treatment composition;
about 0.01 to about 15 wt. %, preferably about 0.1 to about 12 wt. %, more preferably about 0.2 to about 10 wt. % of the at least one alkaline agent selected from organic amines such as an alkanolamine, alkali metal hydroxides, alkali earth metal hydroxides, alkali metal carbonates, alkali metal phosphates, an alkali metal citrate, and mixtures thereof, preferably an alkanolamine;
optionally, about 0.1 to about 15 wt. %, preferably, 0.5 to about 10 wt. %, and more preferably about 1 to about 6 wt. % of at least one cationic surfactant selected from optionally polyoxyalkenylated primary, secondary, tertiary fatty amine salts, quaternary ammonium compounds and their salts, and mixtures thereof, and preferably quaternary ammonium compounds and their salts, and more preferably cetrimonium chloride, behentrimonium chloride, dipalmitoylethylhydroxyethylmonium methosulfate, behentrimonium methosulfate, and mixtures thereof;
optionally, about 0.1 to about 15 wt. %, preferably about 0.5 to about 12 wt. %, more preferably about 1 to about 10 wt. % of at least one nonionic surfactant selected from fatty alcohols, alkoxylated fatty alcohols, alkyl(ether)phosphates, alkylpolyglucosides, fatty acid alkanolamides, and mixtures thereof;
optionally, about 0.01 to about 10, preferably about 0.05 to about 8, more preferably about 0.1 to about 6 wt. % of the at least one thickening agent selected from cellulose polymers, gums, modified or unmodified carboxyvinyl polymers, polyacrylamides, copolymers of acrylic acid and of acrylamide, sodium salts of polyhydroxycarboxylic acids, optionally crosslinked and/or neutralized 2-acrylamido-2-methylpropanesulphonic acid polymers and copolymers, polyacrylic acid/alkyl acrylate, glucans, modified or unmodified starches, silicas, and mixtures thereof;
optionally, about 0.1 to about 15 wt. %, preferably about 0.5 to about 10 wt. %, more preferably about 1 to about 5 wt. % of the least one silicone compound selected from dimethicone; aminosilicones such as amodimethicone, trideceth-9 PG amodimethicone, PEG-40/PPG-8 methylaminopropyl/hydroxypropyl dimethicone copolymer; dimethicone copolyol selected from oxypropylenated and/or oxyethylenated polydimethyl(methyl)siloxane, oxypropylenated and/or oxyethylenated polymethyl (C8-C22) alkyl dimethyl methyl siloxane, and mixtures thereof; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4, including all ranges and sub ranges therebetween.

In one embodiment of the present disclosure, the hair treatment composition includes:
about 0.1 to about 20 wt. %, preferably about 0.5 to about 16 wt. %, more preferably about 1 to about 14 wt. % of thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of the at least non-polymeric and non-thiol, mono-, di-, and/or tricarboxylic acid, a salts thereof, or a mixture thereof, preferably glutaric acid, citric acid, lactic acid, malic acid, salts thereof, or a mixture thereof;
optionally, about 0.01 to about 15 wt. %, preferably about 0.1 to about 12 wt. %, more preferably about 0.2 to about 10 wt. % of the at least one alkaline agent, wherein the at least one alkaline agent is an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine and/or aminomethyl propanol;
optionally, about 0.1 to about 15 wt. %, preferably, 0.5 to about 10 wt. %, and more preferably about 1 to about 6 wt. % of at least one cationic surfactant, wherein the at least one cationic surfactant is a quaternary ammonium compound such as cetrimonium chloride, behentrimonium chloride, dipalmitoylethylhydroxyethylmonium methosulfate, behentrimonium methosulfate, or a mixture thereof;
optionally, about 0.1 to about 15 wt. %, preferably about 0.5 to about 12 wt. %, more preferably about 1 to about 10 wt. % of at least one nonionic surfactant selected from fatty alcohols, alkoxylated fatty alcohols, alkyl(ether)phosphates, alkylpolyglucosides, fatty acid alkanolamides, and mixtures thereof;
optionally, about 0.01 to about 10, preferably about 0.05 to about 8, more preferably about 0.1 to about 6 wt. % of the at least one thickening agent, wherein the at least one thickening agent is a gum, for example, a gum selected from xanthan gum, guar gum, biosaccharide gum, acacia gum, seneca gum, sclerotium gum, gellan gum, and a mixture thereof;
optionally, about 0.1 to about 15 wt. %, preferably about 0.5 to about 10 wt. %, more preferably about 1 to about 5 wt. % of the least one silicone compound selected from dimethicone, amodimethicone, and a mixture thereof; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4.0, including all ranges and sub ranges therebetween.

In one embodiment of the present disclosure, the hair treatment composition includes:
about 0.1 to about 20 wt. %, preferably about 0.5 to about 16 wt. %, more preferably about 1 to about 14 wt. % of at least one thiol-based compound selected from thiolactic acid, an ester thereof, a salt thereof, and a mixture thereof, preferably thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of at least one non-polymeric and non-thiol, monocarboxylic acid, preferably chosen from lactic acid;
about 0.01 to about 15 wt. %, preferably about 0.1 to about 12 wt. %, more preferably about 0.2 to about 10 wt. % of the at least one alkaline agent selected from organic amines such as an alkanolamine, alkali metal hydroxides, alkali earth metal hydroxides, alkali metal carbonates, alkali metal phosphates, an alkali metal citrate, and mixtures thereof, preferably an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine and/or aminomethyl propanol; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4.0, including all ranges and sub ranges therebetween.

In one embodiment of the present disclosure, the hair treatment composition includes:
about 0.1 to about 20 wt. %, preferably about 0.5 to about 16 wt. %, more preferably about 1 to about 14 wt. % of at least one thiol-based compound selected from thiolactic acid, an ester thereof, a salt thereof, and a mixture thereof, preferably thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of at least one non-polymeric and non-thiol, dicarboxylic acid, preferably chosen from malic acid, glutaric acid, a salt thereof, or a mixture thereof, based on the total weight of the hair treatment composition;
about 0.01 to about 15 wt. %, preferably about 0.1 to about 12 wt. %, more preferably about 0.2 to about 10 wt. % of the at least one alkaline agent selected from organic amines such as an alkanolamine, alkali metal hydroxides, alkali earth metal hydroxides, alkali metal carbonates, alkali metal phosphates, an alkali metal citrate, and mixtures thereof, preferably an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine and/or aminomethyl propanol; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4.0, including all ranges and sub ranges therebetween.

In one embodiment of the present disclosure, the hair treatment composition includes:
about 0.1 to about 20 wt. %, preferably about 0.5 to about 16 wt. %, more preferably about 1 to about 14 wt. % of at least one thiol-based compound selected from thiolactic acid, an ester thereof, a salt thereof, and a mixture thereof, preferably thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of at least one non-polymeric and non-thiol, tricarboxylic acid, a salt thereof, or a mixture thereof, preferably chosen from citric acid, based on the total weight of the hair treatment composition;
about 0.01 to about 15 wt. %, preferably about 0.1 to about 12 wt. %, more preferably about 0.2 to about 10 wt. % of the at least one alkaline agent selected from organic amines such as an alkanolamine, alkali metal hydroxides, alkali earth metal hydroxides, alkali metal carbonates, alkali metal phosphates, an alkali metal citrate, and mixtures thereof, preferably an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine and/or aminomethyl propanol; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4.0, including all ranges and sub ranges therebetween.

In one embodiment of the present disclosure, the hair treatment composition includes:
about 0.1 to about 20 wt. %, preferably about 0.5 to about 16 wt. %, more preferably about 1 to about 14 wt. % of at least one thiol-based compound selected from thiolactic acid, an ester thereof, a salt thereof, and a mixture thereof, preferably thiolactic acid;
about 0.1 to about 20 wt. %, preferably about 0.5 to about 15 wt. %, more preferably about 1 to about 12 wt. %, even more preferably about 2 to about 11 wt. %, and most preferably about 3 to about 10 wt. % of at least one non-polymeric and non-thiol, monocarboxylic acid, a salt thereof, or a mixture thereof, preferably chosen from lactic acid, and of at least one non-polymeric and non-thiol, dicarboxylic acid, a salt thereof, or a mixture thereof, preferably chosen from malic acid, glutaric acid, a salt thereof, or a mixture thereof, based on the total weight of the hair treatment composition;
about 0.01 to about 15 wt. %, preferably about 0.1 to about 12 wt. %, more preferably about 0.2 to about 10 wt. % of the at least one alkaline agent selected from organic amines such as an alkanolamine, alkali metal hydroxides, alkali earth metal hydroxides, alkali metal carbonates, alkali metal phosphates, an alkali metal citrate, and mixtures thereof, preferably an alkanolamine selected from monoethanolamine, diethanolamine, triethanolamine, aminomethyl propanol, tris(hydroxymethyl)aminomethane, tetra hydroxy propyl ethylenediamine, a mixture thereof, preferably triethanolamine and/or aminomethyl propanol; and
water;

wherein the pH of the composition is from about 2 to less than 7. In some embodiments, the pH may be from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4.0, including all ranges and sub ranges therebetween.

In one embodiment, the present disclosure is directed to a method of treating hair, the method comprising a treatment cycle, the treatment cycle comprising:

a) optionally, washing the hair with a shampoo and/or rinsing the hair with water, and optionally drying the hair at a temperature ranging from room temperature to about 200° C. (for example, with blow drying) and optionally applying a smoothing action on the hair;

b) applying a hair treatment composition according to the instant disclosure onto the hair and allowing the hair treatment composition to remain on the hair for a period of time ranging from about 1 minute to about 1 hour, from about 15 minutes to about 45 minutes, or for about 30 minutes;

c) after allowing the hair treatment composition to remain on the hair for a period of time, optionally rinsing the hair treatment composition from the hair (preferably, the hair treatment composition is rinsed from the hair but rinsing it from the hair is not required);

d) drying the hair at a temperature ranging from room temperature up to about 200° C., for example, using a blow dryer, while optionally applying a smoothing action on the hair;

e) passing a flat iron over the hair, for example, passing the flat iron over the hair at least once (or at least 2 times, at least 4 times, at least 5 times, or at least 10 times or more, for example, up to 20, 22, 25, 28, 30, 40, 50, 75, or 100 times);

f) after passing the flat iron over the hair, optionally applying a neutralizing composition to the hair (for example, a neutralizing composition comprising hydrogen peroxide) and allowing the neutralizing composition to remain on the hair for a period of time ranging from about 1 minute to about 1 hour, about 5 minutes to about 30 minutes, or about 10 minutes;

g) washing the hair with a shampoo and/or a conditioner and/or rinsing the hair with water; and

h) drying the hair at a temperature ranging from room temperature up to about 200° C., for example, using a blow dryer, while optionally applying a smoothing action on the hair.

In one embodiment, the flat iron is employed at a temperature ranging from about 100° C. to about 250° C. or from about 110° C. to about 230° C. or from about 110° C. to about 210° C. or from about 120° C. to about 200° C. or from about 150° C. to about 190° C., or from about 190° C. to about 250° C., and preferably, at about 190° C., or at about 200° C., or at about 230° C., or at about 250° C. In one embodiment, the flat iron in step (f) is passed over the hair at least 3 times, or at least 5 times, or at least 10 times, or more (for example, up to 20, 22, 25, 28, 30, or times).

In one embodiment, the above-described method of treating hair includes a method of straightening or curl-relaxing hair.

In an embodiment, after passing the flat iron over the hair, the above-described method includes a step of applying a neutralizing composition to the hair (for example, a neutralizing composition comprising an oxidizing agent such as hydrogen peroxide) and allowing the neutralizing composition to remain on the hair for a period of time ranging from about 1 minute to about 1 hour; about 5 minutes to about 30 minutes, or about 10 minutes.

In some embodiments, the at least one oxidizing agent in the neutralizing composition can be selected from hydrogen peroxide, persalts such as persulphates, percarbonates, and perborates, urea peroxide, alkaline bromates, and polythionates. In some embodiments, hydrogen peroxide is used.

In some embodiments, the at least one oxidizing agent in the neutralizing composition may be present in an amount ranging from 0.1 to 50 percent by weight, such as from 1 to 20 percent by weight, relative to the total weight of the composition.

In some embodiments, when the at least one oxidizing agent is hydrogen peroxide, the neutralizing composition described herein may comprise at least one agent that stabilizes hydrogen peroxide. As examples of agents that stabilize hydrogen peroxide, non-limiting mention may be made of the pyrophosphates of the alkali or alkaline-earth metals, the stannates of the alkali or alkaline-earth metals, and phenacetin or the salts of acids and of oxyquinoline, such as oxyquinoline sulphate. In some embodiments, the at least one agent that stabilizes hydrogen peroxide may be present in an amount ranging from 0.0001 to 5 percent by weight, such as from 0.01 to 2 percent by weight, relative to the total weight of the composition.

The above-described method may further comprise a step of rinsing the hair with water after contacting the hair with the neutralizing composition.

The methods described in the present disclosure, including the methods described above, may include imparting to the hair one or more hair care effects selected from:

(i) conditioning;

(ii) straightening or relaxing effects;

(iii) manageability;

(iv) frizz control;

(v) volume reduction or volume control;

(vi) styling or shaping effects;

(vii) curling effects;

(viii) texlaxing effects (resulting in wavy hair or less tight curls);

(ix) improvement or retention of curl definition;

(x) humidity resistance;

(xi) cosmeticity to the feel;

(xii) smooth feel;

(xiii) natural feel;

(xiv) discipline;

(xv) regularity;

(xvi) less or reduced rough ends; and

(xvii) improvement of the appearance of hair

Methods for achieving the one or more effects outlined above comprise applying a hair treatment composition according to the instant disclosure to the hair, and allowing the composition to remain on the hair while styling or straightening the hair thereafter. In one embodiment, the hair treatment composition is applied to the hair more than once over a period of days, for example a period of 2 to 30 days, a period of 2 to 15 days, a period of 2 to 10 days, etc.

In accordance with the present disclosure, the inventors surprisingly and unexpectedly discovered that the hair treatment compositions of the present disclosure effectively condition the hair, and at the same time, provide other hair care benefits to hair fibers such as light weight feel to the hair, straightening or relaxing effects, volume reduction, frizz control, curl reduction, manageability, discipline, cosmeticity, and smooth feel.

The hair treatment compositions of the present disclosure do not require film forming polymers. Therefore, in some embodiments, film forming polymers may be excluded from the hair treatment compositions. In other words, the hair treatment compositions of the instant disclosure may be free or essentially free of film forming polymers.

It was also surprisingly and unexpectedly discovered that the application of the hair treatment compositions to hair results in effectively conditioned hair and imparts/improves the cosmetic properties of hair fibers, in particular human hair, for example, in terms of manageability, straightening or relaxing effects, curl reduction, volume reduction, frizz control, manageability, discipline, cosmeticity, and smooth feel.

The hair treatment compositions described in the present disclosure may be used on any type of hair, for example, light or dark hair, straight or curly, natural hair, or hair that has undergone a cosmetic treatment such as permanent waving, dyeing, coloring, highlighting, bleaching and/or relaxing.

In a preferred embodiment, the composition of the present disclosure is applied on curly, embrittled, and/or damaged hair.

Other subjects and characteristics, aspects and advantages of the disclosure will emerge even more clearly on reading the entirety of the present disclosure.

### Reducing Agents

The hair treatment compositions of the instant disclosure include at least one reducing agent selected from thiol-based compounds, non-thiol-based compounds, and mixtures thereof.

### Thiol-Based Compounds

The thiol-based compounds can be selected from thiolactic acid and derivatives thereof, 3-mercaptopropionic acid and derivatives thereof, cysteamine and derivatives thereof, cysteine and derivatives thereof, thioglycolic acid and derivatives thereof (including esters thereof), diammonium dithiodiglycolate, and mixtures thereof.

In some embodiments, the at least one reducing agent is a thiol-based compound selected from thiolactic acid, glycerol monothiolactate, ammonium thiolactate, MEA-thiolactate, 3-mercaptopropionic acid, glycerol 3-mercaptopropionate, ethyleneglycol 3-mercaptopropionate, cysteamine, N-acetylcysteamine, N-propionylcysteamine, cysteine, N-acetylcysteine, N-alkanoylcysteine, cysteine alkyl esters, homocysteine, thioglycolic acid, ethanolamine thioglycolate, glyceryl thioglycolate, glutathione, thioglycerol, thiomalic acid, thiodiglycol, 2-mercaptoethanol, dithiothreitol, thioxanthine, thiosalicylic acid, thiopropionic acid, lipoic acid, and mixtures thereof.

In an embodiment, the at least one reducing agent is selected from thiolactic acid and derivatives thereof such as an ester thereof, an amide thereof, a salt thereof, and a mixture thereof.

The at least one reducing agent may be selected from thiolactic acid, an ester thereof, an amide thereof, a salt thereof, and a mixture thereof and can be used in combination with other thiol-based compounds selected from thioglycolic acid, cysteine, cysteamine, homocystine, glutathione, thioglycerol, thiomalic acid, 2-mercaptopropionic acid, 3-mercaptopropionic acid, thiodiglycol, 2-mercaptoethanol, dithiothreitol, thioxanthine, thiosalicylic acid, thiopropionic acid, lipoic acid, N-acetylcysteine, their salts thereof, and mixtures thereof.

In an embodiment, the at least one reducing agent can be selected from non-thiol based compounds such as alkali metal, alkaline-earth metal sulfites, hydrides or phosphines, and mixtures thereof.

In some embodiments, the at least one reducing agent can comprise thiol-based compounds and non-thiol-based compounds.

In certain embodiments, the at least one reducing agent used in the composition of the disclosure is thiolactic acid.

### Non-Thiol-Based Compounds

Non-limiting examples of non-thiol-based reducing compounds include ortho-diphenol derivatives having reducing properties. In a manner known per se, the term “ortho-diphenol” denotes compounds comprising at least one aromatic ring, preferably a benzene ring, comprising at least two hydroxyl (OH) groups borne by two adjacent carbon atoms of the aromatic ring which in addition do not comprise a mercapto or disulfide group.

The aromatic ring may more particularly be a fused aryl or fused heteroaromatic ring, i.e. optionally comprising one or more heteroatoms, such as benzene, naphthalene, tetrahydronaphthalene, indane, indene, anthracene, phenanthrene, isoindole, indoline, isoindoline, benzofuran, dihydrobenzofuran, chroman, isochroman, chromene, isochromene, quinoline, tetrahydroquinoline and isoquinoline, said aromatic ring comprising at least two hydroxyl groups borne by two adjacent carbon atoms of the aromatic ring. Preferentially, the aromatic ring of the ortho-diphenol derivatives according to the invention is a benzene ring.

The term “fused ring” means that at least two saturated or unsaturated and heterocyclic or non-heterocyclic rings have a common bond, i.e. that at least one ring is joined side by side with another ring.

The ortho-diphenols according to the invention may or may not be salified. They may also be in aglycone form (without bonded sugars) or in the form of glycosylated compounds.

More particularly, the ortho-diphenol derivative represents a compound of formula (I), or an oligomer thereof, in salified or non-salified form:

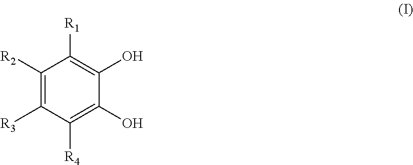

in which formula (I) the substituents:

R₁ to R₄, which may be identical or different, represent: a hydrogen atom, a halogen atom, a hydroxyl radical, a carboxyl radical; an alkyl carboxylate or alkoxycarbonyl radical, an optionally substituted amino radical, an optionally substituted and linear or branched alkyl radical, an optionally substituted and linear or branched alkenyl radical, an optionally substituted cycloalkyl radical, an alkoxy radical, an alkoxyalkyl radical, an alkoxyaryl radical, the aryl group possibly being optionally substituted, an aryl radical, a substituted aryl radical, a saturated or unsaturated heterocyclic radical carrying or not carrying a cationic or anionic charge, optionally substituted and/or optionally fused with an aromatic ring, preferably a benzene ring, said aromatic ring being optionally substituted, in particular with one or more hydroxyl or glycosyloxy groups, a radical containing one or more silicon atoms, or two of the substituents carried by two adjacent carbon atoms R₁-R₂, R₂-R₃ or R₃-R₄ form, together with the carbon atoms carrying them, a saturated or unsaturated and aromatic or non-aromatic ring, optionally comprising one or more heteroatoms and optionally fused with one or more saturated or unsaturated rings optionally comprising one or more heteroatoms. Particularly, R₁ to R₄ together form from one to four rings.

A specific embodiment of the invention relates to ortho-diphenol derivatives of formula (I), two adjacent substituents R₁-R₂, R₂-R₃ or R₃-R₄ of which cannot form, with the carbon atoms which carry them, a pyrrolyl radical. More particularly, R₂ and R₃ cannot form a pyrrolyl radical fused to the benzene ring bearing the two hydroxyls. The ortho-diphenols of use in the invention can be natural or synthetic. The natural ortho-diphenols include the compounds which may be present in nature and which are reproduced by chemical (semi)synthesis.

The salts of the ortho-diphenols of the invention can be salts of acids or of bases. The acids may be mineral or organic. Preferably, the acid is hydrochloric acid, which results in chlorides.

The bases may be mineral or organic. In particular, the bases are alkaline hydroxides, such as sodium hydroxide, which results in sodium salts.

According to one particular embodiment of the invention, the composition(s) comprise as ingredient one or more synthetic ortho-diphenol derivatives that do not exist in nature.

According to another preferred embodiment of the invention, the compositions and methods for treating hair such as for curl-relaxing and/or straightening hair uses, as non-thiol-based compound, one or more natural ortho-diphenol derivatives.

More particularly, the ortho-diphenols that may be used are in particular: flavanols, such as catechin and epicatechin gallate, flavonols, such as quercetin, anthocyanidins, such as cyanidin, delphinidin or petunidin, anthocyanins or anthocyans, for instance myrtillin, ortho-hydroxybenzoates, for example gallic acid salts, flavones, such as luteolin, hydroxystilbenes, for example 3,3′,4,5′-tetrahydroxystilbene, optionally oxylated (for example glucosylated), 3,4-dihydroxyphenylalanine and the derivatives thereof, 2,3-dihydroxyphenylalanine and the derivatives thereof, 4,5-dihydroxyphenylalanine and the derivatives thereof, dihydroxycinnamates, such as caffeic acid and chlorogenic acid, ortho-polyhydroxycoumarins, ortho-polyhydroxyisocoumarins, ortho-polyhydroxycoumarones, ortho-polyhydroxyisocoumarones, ortho-polyhydroxychalcones, ortho-polyhydroxychromones, ortho-polyhydroxyquinones, ortho-polyhydroxyxanthones, 1,2-dihydroxybenzene and the derivatives thereof, 1,2,4-trihydroxybenzene and the derivatives thereof, 1,2,3-trihydroxybenzene and the derivatives thereof, 2,4,5-trihydroxytoluene and the derivatives thereof, proanthocyanidins and especially the proanthocyanidins A1, A2, B1, B2, B3 and C1, proanthocyanins, tannic acid, ellagic acid, and mixtures of the preceding compounds.

When the dye precursors have D and L forms, the two forms may be used in the compositions according to the invention, as may the racemic mixtures.

According to one embodiment, the natural ortho-diphenols result from extracts of animals, bacteria, fungi, algae or plants, used in their entirety or partially. In particular as regards plants, the extracts are derived from plants or plant parts, such as fruit, including citrus fruit, vegetables, trees or shrubs. Use may also be made of mixtures of these extracts, which are rich in ortho-diphenols as defined above. Preferably, the natural ortho-diphenols of the invention are derived from plants or plant parts. The extracts are obtained by extraction of various plant parts, for instance the root, the wood, the bark, the leaf, the flower, the fruit, the seed, the pod or the peel. Use may also be made of mixtures of plant extracts.

According to a specific embodiment of the invention, the ortho-diphenol derivative(s) are natural extracts rich in ortho-diphenols. According to a preferred form, the ortho-diphenol derivative(s) are solely natural extracts. The natural extracts according to the invention may be in the form of powders or liquids. Preferably, the extracts of the invention are provided in the form of powders.

According to a particular embodiment of the invention, the non-thiol-based reducing compound(s) are chosen from meta-hydroxyphenol derivatives, also known as resorcinols, with reducing properties. In a manner known per se, the term “resorcinols” denotes compounds which comprise at least one aromatic ring, preferably a benzene ring, comprising at least two hydroxyl (OH) groups borne by two carbon atoms which are in the meta position relative to each other, and which also do not comprise any mercapto or disulfide groups.

The aromatic ring may more particularly be a fused aryl or fused heteroaromatic ring, i.e. optionally comprising one or more heteroatoms, such as benzene, naphthalene, tetrahydronaphthalene, indane, indene, anthracene, phenanthrene, isoindole, indoline, isoindoline, benzofuran, dihydrobenzofuran, chroman, isochroman, chromene, isochromene, quinoline, tetrahydroquinoline and isoquinoline, said aromatic ring comprising at least two hydroxyl groups borne by two carbon atoms which are in the meta position relative to each other. Preferentially, the aromatic ring of the resorcinol derivatives according to the invention is a benzene ring.

According to a more particular embodiment of the invention, the non-thiol-based reducing compound(s) present in the composition(s) used according to the invention are chosen from the meta-hydroxyphenol derivatives of formula (II), and also the organic or mineral acid or base salts thereof, and the solvates thereof such as hydrates:

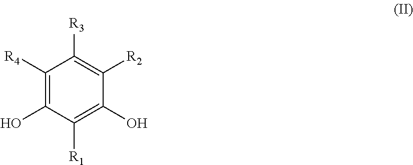

in which formula (II) the substituents: R₁, R₂ and R₄, which may be identical or different, represent: a hydrogen atom, a halogen atom, a carboxyl radical, an alkyl carboxylate or alkoxycarbonyl radical, an optionally substituted amino radical, an optionally substituted and linear or branched alkyl radical, an optionally substituted and linear or branched alkenyl radical, an optionally substituted cycloalkyl radical, an alkylcarbonyl radical, a carboxaldehyde radical, an alkoxy radical, an alkoxyalkyl radical, an alkoxyaryl radical, the aryl group possibly being optionally substituted, an arylalkylcarbonyl radical of which the aryl group, particularly phenyl group, is optionally substituted, preferably with one or more hydroxyl groups, an aryl radical, a substituted aryl radical, a saturated or unsaturated heterocyclic radical optionally bearing a cationic or anionic charge, optionally substituted and/or optionally fused with an aromatic ring, preferably a benzene ring, said aromatic ring being optionally substituted, in particular with one or more hydroxyl or glycosyloxy groups, or a radical containing one or more silicon atoms'

R₃ represents: a hydrogen atom, a halogen atom, a hydroxyl radical, a carboxyl radical, an alkyl carboxylate or alkoxycarbonyl radical, an optionally substituted amino radical, an optionally substituted and linear or branched alkyl radical, a linear or branched alkenyl radical which is optionally substituted, in particular with a phenyl group which is preferably optionally substituted with one or more (di)(C₁-C₄)(alkyl)amino or hydroxyl groups, an optionally substituted cycloalkyl radical, an alkylcarbonyl radical, a carboxaldehyde radical, an alkoxy radical, an alkoxyalkyl radical, an alkoxyaryl radical, the aryl group possibly being optionally substituted, an aryl radical, a substituted aryl radical, a saturated or unsaturated heterocyclic radical optionally bearing a cationic or anionic charge, optionally substituted and/or optionally fused with an aromatic ring, preferably a benzene ring, said aromatic ring being optionally substituted, in particular with one or more hydroxyl or glycosyloxy groups, or a radical containing one or more silicon atoms, or two of the substituents borne by two adjacent carbon atoms R₂-R₃ or R₃-R₄ form, together with the carbon atoms bearing them, a saturated or unsaturated, non-aromatic ring, optionally containing one or more heteroatoms and optionally fused with one or more saturated or unsaturated rings optionally containing one or more heteroatoms. Particularly, R₂ to R₄ together form from two to four rings.

More particularly, the non-thiol-based reducing compound(s) may be chosen from the meta-hydroxyphenols of formula (II) in which the substituents: R₁, R₃ and R₄, which may be identical or different, represent: a hydrogen atom, a halogen atom, a carboxyl radical, an alkyl carboxylate or alkoxycarbonyl radical, an optionally substituted amino radical, an optionally substituted and linear or branched alkyl radical, a linear or branched alkenyl radical which is optionally substituted, in particular with a phenyl group which is preferably optionally substituted with one or more (di)(C₁-C₄)(alkyl)amino or hydroxyl groups, an alkylcarbonyl radical, a carboxaldehyde radical, an alkoxy radical, and/or

R₂ represents: a hydrogen atom, a halogen atom, a hydroxyl radical, a carboxyl radical, an alkyl carboxylate or alkoxycarbonyl radical, an optionally substituted amino radical, a phenylalkylcarbonyl radical of which the phenyl group is optionally substituted, preferably with one or more hydroxyl groups, an optionally substituted and linear or branched alkyl radical, an optionally substituted and linear or branched alkenyl radical, an alkylcarbonyl radical, a carboxaldehyde radical, an alkoxy radical.

More particularly, the substituent R₂ in formula (II) represents a hydrogen atom or a (C₁-C₆)alkyl group, preferably a hydrogen atom.

According to a particular embodiment of the invention, the non-thiol-based reducing compound(s) may be chosen from the resorcinol derivatives of formula (II), and also the organic or mineral acid or base salts thereof, and the solvates thereof such as hydrates: in which

R¹ to R⁴, which may be identical or different, represent a hydrogen or halogen atom, or a group chosen from:
optionally substituted (C₁-C₁₀)alkyl, in particular optionally substituted with at least one hydroxyl radical;
optionally substituted (C₂-C₁₀)alkenyl, in particular optionally substituted with at least one aryl group such as a phenyl group optionally substituted with one or more (di)(C₁-C₄)(alkyl)amino or hydroxyl groups;
(C₁-C₁₀)alkoxy;
carboxy —C(O)—OH or carboxylate —C(O)—O⁻, M⁺; with M⁺ representing a cationic counterion such as an alkali metal or alkaline-earth metal, or an ammonium;
ester —C(O)—O—R⁵ or —O—C(O)—R⁵, with R⁵ representing a (C₁-C₁₀)alkyl group, particularly —C(O)—O—R⁵;
amido —C(O)—NR⁶R⁷ or —NR⁶—C(O)—R⁷ with R⁶ and R⁷, which may be identical or different, representing a hydrogen atom or a (C₁-C₁₀)alkyl group, particularly —C(O)—NH₂;
(C₁-C₁₀)alkylcarbonyl;
hydroxyl; and
amino —NR⁸R⁹, with R⁸ and R⁹, which may be identical or different, representing a hydrogen atom or a (C₁-C₁₀)alkyl group, particularly —NH₂.

In particular, in formula (II), R₁, R₂ and R₄ represent a hydrogen atom or an optionally substituted (C₁-C₆)alkyl group, preferably hydrogen, and/or R₃ represents a hydrogen atom or a linear or branched (C₁-C₆)alkyl or (C₂-C₆)alkenyl group, such as ethenyl, optionally substituted with an aryl group, such as a phenyl group, which is optionally substituted, preferably with one or more (di)(C₁-C₄)(alkyl)amino or hydroxyl groups.

More preferentially, the non-thiol-based reducing compound(s) present in the composition(s) used according to the invention are chosen from the resorcinol derivatives of formula (II), in which R₁, R₂ and R₄ represent a hydrogen atom or a (C₁-C₄)alkyl group, preferably hydrogen, and R₃ represents a hydrogen atom or an Ar—CH═CH— group with Ar representing an aryl group, in particular a phenyl group, said aryl group being optionally substituted, preferably with one or more hydroxyl groups; in particular, said hydroxyl group(s) are substituted in the ortho or para position relative to the phenyl group.

Preferably, the meta-hydroxyphenols according to the invention do not comprise two hydroxyl groups borne by two adjacent carbons.

The meta-hydroxyphenols of the invention may be natural or synthetic. Among the natural meta-hydroxyphenols are included compounds that may be present in nature and that are reproduced by chemical (semi)synthesis. The salts of the meta-hydroxyphenols of the invention may be salts of acids or of bases. The acids may be mineral or organic. Preferably, the acid is hydrochloric acid, which results in chlorides. The bases may be mineral or organic. In particular, the bases are alkaline hydroxides, such as sodium hydroxide, which results in sodium salts.

According to a particular embodiment of the invention, the composition comprises, as ingredient a), one or more synthetic meta-hydroxyphenol derivatives that do not exist in nature.

According to one embodiment, the natural meta-hydroxyphenols are derived from extracts of animals, bacteria, fungi, algae, plants and fruits, used in their entirety or partially. In particular regarding plants, the extracts are derived from fruits, including citrus fruits, from vegetables, from trees and from shrubs. Use may also be made of mixtures of these extracts, which are rich in meta-hydroxyphenols as defined above.

According to a particular embodiment of the invention, the non-thiol-based reducing compound(s) may be chosen from para-hydroxyphenol derivatives with reducing properties. In a manner known per se, the term “para-hydroxyphenol” denotes compounds which comprise at least one aromatic ring, preferably a benzene ring, comprising at least two hydroxyl (OH) groups borne by two adjacent carbon atoms which are in the para position relative to each other, and which also do not comprise any mercapto or disulfide groups.

According to a more particular embodiment of the invention, the non-thiol-based reducing compound(s) may be chosen from the para-hydroxyphenol derivatives of formula (III), and also the organic or mineral acid or base salts thereof, and the solvates thereof such as hydrates:

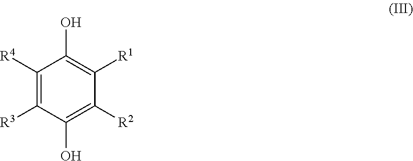

in which formula (III) R¹ to R⁴, which may be identical or different, are as defined previously for formulae (I) and (II), and preferably represent a hydrogen atom or an optionally substituted (C₁-C₄)alkyl group, preferably hydrogen.

Preferentially, the non-thiol-based agent(s) are chosen from catechol, gallic acid, para-hydroxyphenol or resveratrol, it being understood that, when the aromatic ring of the ortho-diphenols, meta-hydroxyphenols or para-hydroxyphenols bear more than two hydroxyl groups (for example three, four, etc.), the compounds should be understood according to the following rule:

If three hydroxyl groups are adjacent on the aromatic ring: (position 1, 2, 3 for example), then said non-thiol-based reducing compound will be considered to be an ortho-diphenol. If there are three hydroxyl groups, two of which are adjacent on the aromatic ring and one is opposite (positions 1, 2, 4 or 1, 2, 5), then said non-thiol-based reducing agent will be considered to be a para-hydroxyphenol. If there are three hydroxyl groups, none of which is adjacent (position 1, 3, 5), then said non-thiol-based reducing compound will be considered to be a meta-hydroxyphenol.

The non-thiol-based reducing agents may be also chosen from phosphines. As examples of phosphines, mention may be made of monophosphines or diphosphines as described in the FR2870119 series.

The at least one reducing agent can be employed in the hair treatment compositions of the present disclosure in an amount of from about 0.1% to about 20% by weight, preferably from about 0.5% to about 16% by weight, more preferably from about 1% to about 14% by weight, or from about 3% to about 14% by weight, more preferably from about 1% to about 12% by weight, or from about 2% to about 11% by weight, or from about 3% to about 11% by weight, or from about 4% to about 11% by weight, or from about 5% to about 11% by weight, relative to the total weight of the composition, including all ranges and subranges therebetween.

In certain embodiments, the at least one reducing agent is thiolactic acid and/or its salts or a mixture thereof, and is employed in the composition of the present disclosure in an amount of about 1%, 1.25%, 1.5%, 1.75%, 2%, 2.25%, 2.5%, 2.75%, 3%, 3.25%, 3.5%, 3.75%, 4%, 4.25%, 4.25%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.5%, 10%, 10.5%, 11%, 11.5%, 12%, 13%, or 14% by weight, relative to the total weight of the composition.

### Non-Polymeric and Non-Thiol, Mono-, Di-, and Tricarboxylic Acid

The hair treatment compositions of the instant disclosure include at least one non-polymeric and non-thiol, mono-, di-, and/or tricarboxylic acids, and/or salts thereof, or a mixture thereof. In one embodiment, the non-polymeric mono, di, and tricarboxylic acids have a molecular weight of less than about 500 g/mol, preferably less than about 400 g/mol, and even more preferably less than about 300 g/mol.

In one embodiment, the hair treatment compositions include at least one monocarboxylic acid. For example, the hair treatment compositions may include at least one monocarboxylic acid selected from lactic acid, formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof. In some instances, lactic acid and/or a salt thereof is a preferred monocarboxylic acid.

In one embodiment, the hair treatment compositions include at least one dicarboxylic acid. For example, the hair treatment compositions may include at least one dicarboxylic acid selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof. In some instances, malic acid, glutaric acid, a salt thereof, or a mixture thereof are preferred dicarboxylic acids.

In one embodiment, the hair treatment compositions include at least one tricarboxylic acid. For example, the hair treatment compositions may include at least one tricarboxylic acid selected from citric acid, isocitric acid, aconitric acid, propane-1,2,3-tricarboxylic acid, benzene-1,3,5-tricarboxylic acid, a salt thereof, and a mixture thereof. In some instances, citric acid and/or a salt thereof are preferred tricarboxylic acids.

In one embodiment, the hair treatment compositions include at least two non-polymeric and non-thiol, mono-, di-, and/or tri-carboxylic acids, and/or salts thereof. The at least two non-polymeric and non-thiol, mono-, di-, and/or tric-arboxylic acids may be, for example, any two or more of those listed above.

In one embodiment, the hair treatment compositions include at least two dicarboxylic acids and/or salts thereof. For example, the at least two dicarboxylic acids and/or salts thereof may be selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof; preferably the at least two dicarboxylic acids include glutaric acid, malic acid, and/or salts thereof.

In one embodiment, the hair treatment compositions include at least one monocarboxylic acid and/or salts thereof and at least one dicarboxylic acid and/or salts thereof. For example, the at least one monocarboxylic acid and/or salts thereof may be selected from lactic acid, formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof, and the at least one dicarboxylic acid and/or salts thereof may be selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof; preferably, the at least one monocarboxylic acid includes lactic acid and/or salts thereof, and the at least one dicarboxylic acid includes glutaric acid, malic acid, and/or salts thereof.

In one embodiment, the hair treatment compositions include at least one tricarboxylic acid and/or salts thereof and at least one dicarboxylic acid and/or salts thereof. For example, the at least one tricarboxylic acid and/or salts thereof may be selected from citric acid, isocitric acid, aconitric acid, propane-1,2,3-tricarboxylic acid, benzene-1,3,5-tricarboxylic acid, a salt thereof, and a mixture thereof, and the at least one dicarboxylic acid and/or salts thereof may be selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof; preferably the at least one tricarboxylic acid includes citric acid and/or salts thereof, and the at least one dicarboxylic acid includes glutaric acid, malic acid, and/or salts thereof.

In one embodiment, the hair treatment compositions include at least one tricarboxylic acid and/or salts thereof and at least one monocarboxylic acid and/or salts thereof. For example, the at least one tricarboxylic acid and/or salts thereof may be selected from citric acid, isocitric acid, aconitric acid, propane-1,2,3-tricarboxylic acid, benzene-1,3,5-tricarboxylic acid, a salt thereof, and a mixture thereof, and the at least one monocarboxylic acid and/or salts thereof may be selected from lactic acid, formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, a salt thereof, and a mixture thereof; preferably the at least one monocarboxylic acid includes lactic acid and/or salts thereof, and the at least one dicarboxylic acid includes glutaric acid, malic acid, and/or salts thereof.

In certain embodiments, the total amount of the at least one non-polymeric and non-thiol, mono-, di-, and/or tricarboxylic acids, and/or salts, or a mixture thereof in the hair treatment compositions is from about 0.1% to about 20% by weight, preferably from about 0.5% to about 15% by weight, more preferably from about 1% to about 12% by weight, even more preferably from about 2% to about 11% by weight, and even more preferably from about 3% to about 10% by weight, and most preferably from about 5% to about 10% by weight, based on the total weight of the hair treatment composition.

In certain embodiments, the at least one non-polymeric and non-thiol, mono-, di-, and tricarboxylic acids, and/or salts thereof, or a mixture thereof can be employed in the hair treatment compositions of the present disclosure in an amount of about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20% by weight, relative to the total weight of the hair treatment composition.

### Alkaline Agent

The hair treatment compositions of the present disclosure include at least one alkaline agent, for example, at least one alkaline agent selected from organic amines, ammonium salts, inorganic and hydroxide bases such as alkali metal hydroxides, alkali earth metal hydroxides, alkali metal carbonates, alkali metal phosphates, alkali metal citrates, and mixtures thereof.

Organic amines may be chosen from organic amines comprising one or two primary, secondary, or tertiary amine functions, and at least one linear or branched C₁-C₈ alkyl groups bearing at least one hydroxyl radical.

Organic amines may also be selected cyclic amines and other cyclic compounds, saturated or unsaturated, having one or more nitrogen atoms within the ring, and mixtures thereof.

The organic amines may be chosen from the ones having a pKb at 25° C. of less than 12, such as less than 10 or such as less than 6. It should be noted that this is the pKb corresponding to the function of highest basicity.

Organic amines may also be chosen from alkanolamines such as mono-, di- or trialkanolamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof.

Among the compounds of the alkanolamine type that may be mentioned include but not limited to: monoethanolamine (also known as monoethanolamine or MEA), diethanolamine, triethanolamine, monoisopropanolamine, aminomethyl propoanol, diisopropanolamine, triisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol (aminomethyl propanol or AMP), tris(hydroxymethylamino)methane, tetrahydroxypropyl ethylenediamine, tromethamine, a mixture thereof, preferably triethanolamine and/or aminomethyl propanol.

Other examples include but are not limited to: 1,3-diaminopropane, 1,3-diamino-2-propanol, spermine, and spermidine.

In some embodiments, the organic amines are chosen from amino acids.

As non-limiting examples, the amino acids that may be used may be of natural or synthetic origin, in L, D, or racemic form, and comprise at least one acid function chosen from, for instance, carboxylic acid, sulfonic acid, phosphonic acid, and phosphoric acid functions. The amino acids may be in their neutral or ionic form.

Further as non-limiting examples, the amino acids may be chosen from basic amino acids comprising an additional amine function optionally included in a ring or in a ureido function.

Amino acids that may be used in the present disclosure include but are not limited to: aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, ornithine, citrulline, and valine.

In some embodiments, the organic amines are chosen from organic amines of heterocyclic type. Besides histidine that has already been mentioned in the amino acids, non-limiting mention may also be made of pyridine, piperidine, imidazole, 1,2,4-triazole, tetrazole, and benzimidazole.

In some embodiments, the organic amines are chosen from amino acid dipeptides. Amino acid dipeptides that may be used in the present disclosure include but not limited to: carnosine, anserine, and baleine.

In some embodiments, the organic amines are chosen from compounds comprising a guanidine function. Organic amines of this type that may be used in the present disclosure include, besides arginine that has already been mentioned as an amino acid, creatine, creatinine, 1,1-dimethylguanidine, 1,1-diethylguanidine, glycocyamine, metformin, agmatine, N-amidinoalanine, 3-guanidinopropionic acid, 4-guanidinobutyric acid, and 2-([amino(imino)methyl]amino)ethane-1-sulfonic acid.

In some embodiments, the organic amines are chosen from alkylamines such as monoamines, diamines, triamines, or mixtures thereof. An example of diamines that can be used as alkaline agents of the invention are 2,2′-(ethylenedioxy)bis(ethylamine) and 4,7,10-trioxa-1,13-tridecanediamine.

The organic amine may be in salt form. The term “organic amine salt,” as used herein, means organic or mineral salts of an organic amine as described above.

As a non-limiting example, the organic salts may be chosen from the salts of organic acids, such as citrates, lactates, glycolates, gluconates, acetates, propionates, fumarates, oxalates and tartrates.

In one embodiment, the organic salt is trisodium citrate.

Further as a non-limiting example, the mineral salts may be chosen from hydrohalides (for example hydrochlorides), carbonates, hydrogen carbonates, sulfates, hydrogen phosphates, and phosphates.

The ammonium salts that may be used according to the present disclosure may be chosen from the following acid salts: carbonate, bicarbonate. For instance, the salt is the carbonate, such as ammonium carbonate.

The alkali metal phosphates and carbonates that may be used are, for example, sodium phosphate, potassium phosphate, sodium carbonate, sodium bicarbonate, potassium carbonate, potassium bicarbonate, and their derivatives.

Alkaline agents may also be chosen from inorganic bases and hydroxide bases such as alkali metal hydroxides, alkaline-earth metal hydroxides, transition metal hydroxides, quaternary ammonium hydroxides, organic hydroxides, and mixtures thereof. Suitable examples are ammonium hydroxide, sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and mixtures thereof.

According to at least one embodiment, the at least one alkaline agent is chosen from aminomethyl propanol, sodium hydroxide, potassium hydroxide, lithium hydroxide, aminomethyl propanediol, triisopropanol amine, dimethylstearylamine, dimethyl/tallowamine, lysine, ornithine, arginine, monoethanolamine, triethanolamine, calcium hydroxide, calcium bicarbonate, and mixtures thereof.

According to another preferred embodiment, the at least one alkaline agent is chosen from aminomethyl propanol, sodium hydroxide, lithium hydroxide, calcium hydroxide, potassium hydroxide, ammonia, disodium phosphate, L-lysine, L-arginine, tromethamine, aminomethyl propanol, monoethanolamine, triethanolamine, tetra hydroxy propyl ethylenediamine, triisopropanolamine, trisodiuim citrate, 2,2′-(ethylenedioxy)bis(ethylamine), 4,7,10-trioxa-1,13-tridecanediamine, and mixtures thereof.

According to an especially preferred embodiment, the at least one alkaline agent is chosen from an alkanolamine. Non-limiting examples of particularly useful alkanolamines include monoethanolamine, diethanolamine, triethanolamine, aminomethyl propanol, triisopropanolamine, tromethamine, tris(hydroxymethyl)aminomethane, tetra hydroxy propyl ethylenediamine, a mixture thereof, preferably triethanolamine and/or aminomethyl propanol.

The at least alkaline agent may be present in the hair treatment compositions in an amount of from 0.01% to 15% by weight, preferably from 0.1% to 10% by weight, more preferably from 0.2% to 8% by weight, even more preferably from 0.5% to 5% by weight, relative to the total weight of the composition, including all ranges and sub ranges therebetween.

When the at least alkaline agent is selected from aminomethyl propanol, it may be present in an amount of from 0.1% to 6.3% by weight, preferably from 0.2% to 5.5% by weight, more preferably from 0.3% to 5% by weight, even more preferably from 0.3% to 4.6% by weight, relative to the total weight of the composition, including all ranges and sub ranges therebetween.

When the at least alkaline agent is sodium hydroxide, it may be present in an amount of from 0.1% to 4.1% by weight, preferably from 0.15% to 3.5% by weight, more preferably from 0.2% to 3% by weight, even more preferably from 0.5% to 3% by weight, relative to the total weight of the composition, including all ranges and sub ranges therebetween.

When the at least alkaline agent is an alkanolamine, it may be present in an amount of from 0.1% to 10 by weight, preferably from 0.5 to 8% by weight, more preferably from 0.5% to 6% by weight, even more preferably from 1% to 5% by weight, relative to the total weight of the composition, including all ranges and sub ranges therebetween.

### Cationic Surfactant

The hair treatment compositions according to the present disclosure may include at least one cationic surfactant.

Non-limiting examples of cationic surfactants useful in the disclosure include, for example, optionally polyoxyalkylenated primary, secondary or tertiary fatty amine salts, quaternary ammonium salts, and mixtures thereof.

Quaternary ammonium salts useful in the disclosure include, for example:
quaternary ammonium salts having formula (Ia):

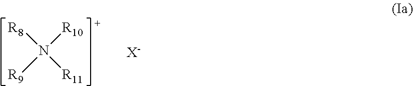

in which:

the groups R8 to R11, which may be identical or different, represent a linear or branched aliphatic group containing from 1 to 30 carbon atoms, or an aromatic group such as aryl or alkylaryl, at least one of the groups R8 to R11 containing from 8 to 30 carbon atoms and preferably from 12 to 24 carbon atoms; it being possible for the aliphatic groups to comprise heteroatoms such as, in particular, oxygen, nitrogen, sulfur or halogens;

The aliphatic groups are chosen, for example, from C1-C30 alkyl, C1-C30 alkoxy, (C2-C6)polyoxyalkylene, C1-C30 alkylamide, (C12-C22)alkyl(C2C6)alkylamido, (C12-C22)alkyl acetate and C1-C30 hydroxyalkyl groups.

X— is an anion chosen from halides, phosphates, acetates, lactates, (C1-C4)alkyl sulfates, (C1-C4)alkylsulfonates and (C1-C4)alkylarylsulfonates.

Among the quaternary ammonium salts having formula (Ia), preference is given to tetraalkylammonium chlorides such as, for example, dialkyldimethylammonium or alkyltrimethylammonium chlorides in which the alkyl group comprises approximately from 12 to 22 carbon atoms, particularly behenyltrimethylammonium, distearyldimethylammonium, cetyltrimethylammonium, and benzyldimethylstearylammonium chlorides, or secondly, to palmitylamidopropyltrimethylammonium chloride or stearamidopropyldimethyl(myristyl acetate)-ammonium chloride, which is sold under the name Ceraphyl® 70 by the company Van Dyk.
imidazoline quaternary ammonium salts having formula (IIa):

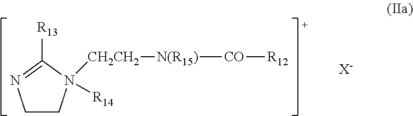

in which

R12 represents an alkenyl or alkyl group comprising from 8 to 30 carbon atoms, for example fatty acid derivatives of tallow;

R13 represents a hydrogen atom, a C1-C4 alkyl group or an alkenyl or alkyl group comprising from 8 to 30 carbon atoms;

R14 represents a C1-C4 alkyl group;

R15 represents a hydrogen atom or a C1-C4 alkyl group;

X— is an anion chosen from halides, phosphates, acetates, lactates, (C1-C4)alkyl sulfates, (C1-C4)alkylsulfonates and (C1-C4)alkylarylsulfonates;

R12 and R13 preferably denote a mixture of alkenyl or alkyl groups containing from 12 to 21 carbon atoms, for example fatty acid derivatives of tallow, R14 denotes a methyl group, and R15 denotes a hydrogen atom. A product of this kind is sold for example under the name Rewoquat® W 75 by the company Rewo.
quaternary di- or triammonium salts having formula (IIIa):

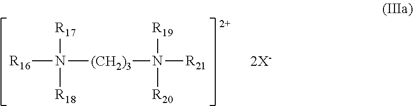

in which

R16 denotes an alkyl group containing approximately from 16 to 30 carbon atoms, which is optionally hydroxylated and/or interrupted with one or more oxygen atoms, R17 is chosen from hydrogen or an alkyl group containing from 1 to 4 carbon atoms or a group —(CH2)3-N+(R16a)(R17a)(R18a);

R16a, R17a, R18a, R18, R19, R20 and R21, which may be identical or different, are chosen from hydrogen and an alkyl group comprising from 1 to 4 carbon atoms; and

X— is an anion chosen from halides, acetates, phosphates, nitrates, (C1-C4)alkyl sulfates, (C1-C4)alkylsulfonates and (C1-C4)alkylarylsulfonates, in particular methyl sulfate and ethyl sulfate.

Such compounds are, for example, Finquat® CT-P, sold by the company Finetex (Quaternium 89), and Finquat® CT, sold by the company Finetex (Quaternium 75),
quaternary ammonium salts containing one or more ester functions having the following formula (IVa):

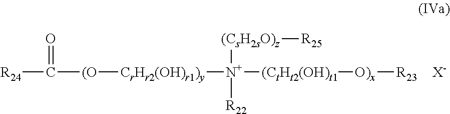

in which:

R22 is chosen from C1-C6 alkyl groups and C1-C6 hydroxyalkyl or C1-C6 dihydroxyalkyl groups; R23 is chosen from the group R26-C(═O)—; hydrocarbon-based linear or branched, saturated or unsaturated C1-C22 groups R27; and a hydrogen atom;

R25 is chosen from the group R28-C(═O)—; hydrocarbon-based linear or branched, saturated or unsaturated C1-C6 groups R29; and a hydrogen atom;

R24, R26 and R28, which may be identical or different, are chosen from linear or branched, saturated or unsaturated C7-C21 hydrocarbon-based groups;

r, s and t, which may be identical or different, are integers ranging from 2 to 6;

r1 and t1, which may be identical or different, are equal to 0 or 1;

r2+r1=2 r and t1+t2=2 t;

y is an integer ranging from 1 to 10;

x and z, which may be identical or different, are integers ranging from 0 to 10;

X— is a simple or complex, organic or mineral anion;
with the proviso that the sum x+y+z is from 1 to 15, that when x is 0 then R23 denotes R27, and that when z is 0 then R25 denotes R29.

The alkyl groups R22 may be linear or branched, and more particularly linear. Preferably, R22 denotes a methyl, ethyl, hydroxyethyl or dihydroxypropyl group, and more particularly a methyl or ethyl group.

Advantageously, the sum x+y+z is from 1 to 10.

When R23 is an R27 hydrocarbon group, it may be long and may have from 12 to 22 carbon atoms, or may be short and may have from 1 to 3 carbon atoms.

When R25 is an R29 hydrocarbon group, it preferably has 1 to 3 carbon atoms.

Advantageously, R24, R26 and R28, which are identical or different, are chosen from linear or branched, saturated or unsaturated C₁₁-C₂₁ hydrocarbon groups, and more particularly from linear or branched, saturated or unsaturated C₁₁-C₂₁ alkyl and alkenyl groups.

Preferably, x and z, which may be identical or different, are equal to 0 or 1. Advantageously, y is equal to 1. Preferably, r, s and t, which may be identical or different, are equal to 2 or 3, and even more particularly are equal to 2.

The anion X— is preferably a halide, preferably chloride, bromide or iodide, a (C₁-C₄)alkyl sulfate, (C₁-C₄)alkyl sulfonate or (C₁-C₄)alkylaryl sulfonate. However, it is possible to use methanesulfonate, phosphate, nitrate, tosylate, an anion derived from an organic acid, such as acetate or lactate, or any other anion that is compatible with the ammonium comprising an ester function.

The anion X— is more particularly still chloride, methyl sulfate or ethyl sulfate.

Use is made more particularly, in the composition according to the disclosure, of the ammonium salts having formula (IVb) in which:
R22 denotes a methyl or ethyl group,
x and y are equal to 1,
z is equal to 0 or 1,
r, s and t are equal to 2,
R23 is chosen from the group R26-C(═O)—; methyl groups, ethyl groups or hydrocarbon-based C14-C22 groups; and a hydrogen atom,
R25 is chosen from the group R28-C(═O)—; and a hydrogen atom,
R24, R26 and R28, which may be identical or different, are chosen from linear or branched, saturated or unsaturated C13-C17 hydrocarbon groups, and preferably from linear or branched, saturated or unsaturated C13-C17 alkyl and alkenyl groups.

The hydrocarbon-based groups are advantageously linear.

Among the compounds of formula (IVb), examples that may be mentioned include salts, in particular the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent, such as an alkyl halide, preferably methyl or ethyl halide, a dialkyl sulfate, preferably methyl or ethyl sulfate, methyl methanesulfonate, methyl para-toluenesulfonate, glycol chlorohydrin or glycerol chlorohydrin. Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the disclosure may contain, for example, a mixture of quaternary ammonium monoester, diester and triester salts with a weight majority of diester salts. Use may also be made of the ammonium salts containing at least one ester function that are described in U.S. Pat. Nos. 4,874,554 and 4,137,180. Use may also be made of behenoylhydroxypropyltrimethylammonium chloride, for example, sold by the company Kao under the name Quartamin BTC 131.

Preferably, the ammonium salts containing at least one ester function.

Preferably, the cationic surfactants are chosen from cetrimonim chloride (cetyltrimethylammonium salt), behentrimonim chloride (behenyltrimethylammonium salt), behentrimonium methosulfate, dipalmitoylethylhydroxyethylmethylammonium salts, and mixtures thereof.

The composition according to the disclosure may comprise the cationic surfactant in an amount ranging from about 0.1% to about 15% by weight, preferably about 0.2% to about 13%, more preferably from about 0.5% to about 12% by weight, and even more preferably from about 1% to about 8% by weight, relative to the total weight of the composition, including all ranges and subranges therebetween. In a particular embodiment, the amount of the at least one cationic surfactant is at about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5%, 5.5%, 6%, 6.5%, 7%, 7.5%, 8%, 8.5%, 9%, 9.5%, or 10% by weight, relative to the total weight of the composition.

### Nonionic Surfactant

In one embodiment, the hair treatment compositions of the present disclosure include at least one nonionic surfactant, for example, at least one nonionic surfactant selected from fatty alcohols, alkoxylated fatty alcohols, alkyl(ether)phosphates, alkylpolyglucosides, fatty acid alkanolamides, and mixtures thereof.

### Fatty Alcohols

The fatty alcohols correspond to linear, branched saturated/unsaturated fatty alcohols comprising from 6 to 60 carbon atoms and preferably correspond to the formula R—OH in which R is a saturated or unsaturated, linear or branched hydrocarbon-based radical, comprising 6 to 60 carbon atoms, or from 10 to 50 carbon atoms, or from 12 to 24 carbon atoms, or from 10 to 22 carbon atoms optionally comprising one or more OH groups.

The saturated fatty alcohols may be branched and can be in liquid form. They can optionally comprise, in their structure, at least one aromatic or non-aromatic ring. They are preferably acyclic.

The unsaturated fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They are preferably acyclic.

Liquid fatty alcohols may be selected, for example, from octyldodecanol, 2-butyloctanol, 2-hexyldecanol, 2-undecylpentadecanol, oleyl alcohol, linoleyl alcohol, isostearyl alcohol, undecylenyl alcohol, linolenyl alcohol and mixtures thereof.

The fatty alcohols of the disclosure may be in solid form and may be non-oxyalkylenated and/or non-glycerolated. These fatty alcohols may be constituents of animal or plant waxes.

The solid fatty alcohol may represent a mixture of fatty alcohols, which means that several species of fatty alcohol may coexist, in the form of a mixture, in a commercial product. One example of such a commercial product is cetearyl alcohol, a mixture of cetyl alcohol and stearyl alcohol, commercially available under the trade name of LANETTE-O from the company BASF. Cetyl alcohol may also be commercially available under the tradename of LANETTE 16 from the company BASF.

In an embodiment, the solid fatty alcohols of the present disclosure may be chosen from myristyl alcohol, cetyl alcohol, stearyl alcohol, cetearyl alcohol, and mixtures thereof, octyldodecanol, 2-butyloctanol, 2-hexyldecanol, 2-undecylpentadecanol, oleic alcohol, linoleic alcohol, behenyl alcohol, and mixtures thereof.

Other suitable examples of the solid fatty alcohol of the present disclosure include branched solid fatty alcohols chosen from 2-dodecylhexadecanol, 2-tetradecyl-1-octadecanol, 2-tetradecyl-1-eicosanol, 2-hexadecyl-1-octadecanol and 2-hexadecyl-1-eicosanol, and mixtures thereof.

In embodiment of the present disclosure, the fatty alcohol comprises cetyl alcohol and stearyl alcohol or cetearyl alcohol.

In one embodiment of the present disclosure, the fatty alcohol is chosen from cetyl alcohol, stearyl alcohol, cetearyl alcohol, and mixtures thereof.

### Alkoxylated Fatty Alcohols

“Alkoxylated fatty alcohol” as used herein means a compound having at least one fatty portion (8 carbon atoms or more) and at least one alkoxylated portion (—(CH₂)ₙO—, where n is an integer from 1 to 5, preferably 2 to 3). According to particularly preferred embodiments, the alkoxylated fatty alcohols of the present disclosure can be used as non-ionic surfactants, if desired. In this regard, the alkoxylated fatty alcohols of the present disclosure preferably have an HLB (hydrophilic-lipophilic balance) value from 1-20, including all ranges and subranges therebetween, with HLB values ranging from 1 to 5 (particularly 3 to 5) or from 15-20 (particularly 16 to 18) being most preferred. Preferably, the alkoxylated fatty alcohol is chosen from ethoxylated fatty alcohols, propoxylated fatty alcohols, and mixtures thereof.

Preferably, the alkoxylated fatty alcohol can be chosen from di-alkyl, tri-alkyl- and combinations of di-alkyl and tri-alkyl substituted ethoxylated polymers. They can also be chosen from mono-alkyl, di-alkyl, tri-alkyl, tetra-alkyl substituted alkyl ethoxylated polymers and all combinations thereof. The alkyl group can be saturated or unsaturated, branched or linear and contain a number of carbon atoms preferably from about 12 carbon atoms to about 50 carbon atoms, including all ranges and subranges therebetween, for example, 20 to 40 carbon atoms, 22 to 24 carbon atoms, 30 to 50 carbon atoms, and 40 to 60 carbon atoms. Most preferably, the fatty portion contains a mixture of compounds of varying carbon atoms such as, for example, C20-C40 compounds, C22-C24 compounds, C30-C50 compounds, and C40-C60 compounds.

Preferably, the alkoxylated portion of the alkoxylated fatty alcohols of the present disclosure contain 2 or more alkoxylation units, preferably from 10 to 200 alkoxylation units, preferably from 20 to 150 alkoxylation units, and preferably from 25 to 100 alkoxylation units, including all ranges and subranges therebetween. Also preferably, the alkoxylation units contain 2 carbon atoms (ethoxylation units) and/or 3 carbon atoms (propoxylation units).

The amount of alkoxylation can also be determined by the percent by weight of the alkoxylated portion with respect to the total weight of the compound. Suitable weight percentages of the alkoxylated portion with respect to the total weight of the compound include, but are not limited to, 10 percent to 95 percent, preferably 20 percent to 90 percent, including all ranges and subranges therebetween with 75 percent to 90 percent (particularly 80 percent to 90 percent) or 20 percent to 50 percent being preferred.

Preferably, the alkoxylated fatty alcohols of the present disclosure have a number average molecular weight (Mn) greater than 500, preferably from 500 to 5,000, including all ranges and subranges therebetween such as, for example, Mn of 500 to 1250 or an Mn of 2,000 to 5,000.

Suitable examples of alkoxylated fatty alcohols include: laureth-3, laureth-7, laureth-9, laureth-12, laureth-23, ceteth-10, ceteth-33, steareth-10, steareth-2, steareth-20, steareth-100, beheneth-5, beheneth-5, beheneth-10, oleth-10, Pareth alcohols, trideceth-10, trideceth-12, C12-13 pareth-3, C12-13 pareth-23, C11-15 pareth-7, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and mixtures thereof.

### Alkyl(Ether)Phosphates

Suitable alkyl(ether)phosphates include, but are not limited to, alkoxylated alkyl phosphate esters and alkyl phosphate esters corresponding to a mono-ester of formula (I) and salts thereof:
RO[CH₂O]ᵤ[(CH₂)ₓCH(R′)(CH₂)_{y}(CH₂)_{z}O]ᵥ[CH₂CH₂O]_{w}—PO—(OH)₂ Formula (I); a di-ester corresponding to formula (II) and salts thereof:
{RO[CH₂O]ᵤ[(CH₂)ₓCH(R′)(CH₂)_{y}(CH₂)_{z}O]ᵥ[CH₂CH₂O]_{w}}₂PO—(OH) Formula (II); a tri-ester corresponding to formula (III):
{RO[CH₂O]ᵤ[(CH₂)ₓCH(R′)(CH₂)_{y}(CH₂)_{z}O]ᵥ[CH₂CH₂O]_{w}}₃PO Formula (III);

and combinations thereof, wherein:

R is a hydrocarbon radical containing from 6 to 40 carbon atoms;

u, v and w, independently of one another, represent numbers of from 0 to 60;

x, y and z, independently of one another, represent numbers of from 0 to 13;

R′ represents hydrogen, alkyl, the sum of x+y+z being ≥0. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formulas (I), (II) and (III), R is linear or branched, acyclic or cyclic, saturated or unsaturated, aliphatic or aromatic, substituted or unsubstituted, preferably a linear or branched, acyclic C₆₋₄₀ alkyl or alkenyl group or a C₁₋₄₀ alkyl phenyl group, more particularly a C₈₋₂₂ alkyl or alkenyl group or a C₄₋₁₈ alkyl phenyl group, more preferably a C₁₂₋₁₈ alkyl group or alkenyl group or a C₆₋₁₆ alkyl phenyl group; u, v, w, independently of one another, is preferably a number from 2 to 20, more preferably a number from 3 to 17 and most preferably a number from 5 to 15;

x, y, z, independently of one another, is preferably a number from 2 to 13, more preferably a number from 1 to 10 and most preferably a number from 0 to 8.

In general, the lower the number of carbon atoms in the R group of the phosphate esters, the more irritating to the skin and the less soluble in water the phosphate ester becomes. In contrast, the higher the number of carbon atoms in the R group, the milder to the skin and the thicker and more waxy the resultant product becomes. Accordingly, for best results, R should have from 12 to 18 carbon atoms.

Particularly preferred alkoxylated alkyl phosphate esters for use in the present disclosure are PPG-5-Ceteth-10 phosphate (CRODAFOS SG(R)), Oleth-3 phosphate (CRODAFOS N3 acid), Oleth-10 phosphate (CRODAFOS N10 acid), and a mixture of Ceteth-10 phosphate and Dicetyl phosphate (CRODAFOS CES) all sold by Croda. Particularly preferred alkyl phosphate esters are Cetyl phosphate (Hostaphat CC 100), Stearyl phosphate (Hostaphat CS 120) from Clariant.

In one embodiment, the alkyl(ether)phosphates are chosen from PPG-5-Ceteth-10 phosphate, Oleth-3 phosphate, Oleth-10 phosphate, Ceteth-10 phosphate, a mixture of Ceteth-10 phosphate and Dicetyl phosphate, Dicetyl phosphate, Cetyl phosphate, Stearyl phosphate, and mixtures thereof.

### Alkylpolyglucosides

The alkyl(poly)glucoside (alkylpolyglycoside) is represented especially by the following general formula:

R₁O—(R₂O)ₜ-(G)ᵥ

wherein:
R₁ represents a linear or branched alkyl or alkenyl radical comprising 6 to 24 carbon atoms and especially 8 to 18 carbon atoms, or an alkylphenyl radical whose linear or branched alkyl radical comprises 6 to 24 carbon atoms and especially 8 to 18 carbon atoms;
R₂ represents an alkylene radical comprising 2 to 4 carbon atoms,
G represents a sugar unit comprising 5 to 6 carbon atoms,
t denotes a value ranging from 0 to 10 and preferably 0 to 4,
v denotes a value ranging from 1 to 15 and preferably 1 to 4.

Preferably, the alkylpolyglycoside surfactants are compounds of the formula described above in which:
R₁ denotes a linear or branched, saturated or unsaturated alkyl radical comprising from 8 to 18 carbon atoms,
R₂ represents an alkylene radical comprising 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C8/C16 alkyl(poly)glycosides 1,4, and especially decyl glucosides and caprylyl/capryl glucosides, are most particularly preferred.

Among the commercial products, mention may be made of the products sold by the company COGNIS under the names PLANTAREN® (600 CS/U, 1200 and 2000) or PLANTACARE® (818, 1200 and 2000); the products sold by the company SEPPIC under the names ORAMIX CG 110 and ORAMIX NS 10; the products sold by the company BASF under the name LUTENSOL GD 70, or else the products sold by the company CHEM Y under the name AG10 LK.

Preferably, use is made of C8/C16-alkyl(poly)glucosides 1,4, especially as an aqueous 53% solution, such as those sold by Cognis under the reference Plantacare® 818 UP.

In an embodiment, the alkylpolyglucoside is chosen from decyl glucoside, stearyl glucoside, lauryl glucoside, coco-glucoside, cetearyl glucoside, decyl lauryl glucoside, and mixtures thereof.

### Fatty Acid Alkanolamides

Suitable fatty acid alkanolamides include those formed by reacting an alkanolamine and a C6-C36 fatty acid. Such surfactants can be chosen from mono-alkanolamides and di-alkanolamides of C6-C36 fatty acids, and preferably from mono-alkanolamides and di-alkanolamides of C8-C30 fatty acids or of C8-C24 fatty acids, and may have a C2-3 hydroxyalkyl group. Examples thereof include, but are not limited to:

oleic acid diethanolamide, oleic acid monoisopropanolamide, myristic acid monoethanolamide, soya fatty acids diethanolamide, stearic acid ethanolamide, linoleic acid diethanolamide, behenic acid monoethanolamide, isostearic acid monoisopropanolamide, erucic acid diethanolamide, ricinoleic acid monoethanolamide, coconut isopropanolamide (INCI name: Cocamide MIPA), coconut fatty acid monoethanolamide (INCI name: Cocamide MEA), coconut fatty acid diethanolamide, palm kernel fatty acid diethanolamide, lauric monoethanolamide, lauric diethanolamide, lauric isopropanolamide polyoxyethylene coconut fatty acid monoethanolamide, and mixtures thereof.

In an embodiment, the fatty acid alkanolaminde is chosen from Cocamide MIPA, Cocamide MEA (Coco monoethanolamide), and mixtures thereof.

In one embodiment of the present disclosure, the at least one nonionic surfactant is selected from cetyl alcohol, stearyl alcohol, cetearyl alcohol (mixture of cetyl alcohol and stearyl alcohol), octyldodecanol, isostearyl alcohol, 2-hexyl decanol, palmityl alcohol, myristyl alcohol, stearyl alcohol, lauryl alcohol, oleic alcohol (or oleyl), linoleyl alcohol (or linoley-ether), linolenic alcohol (or linolenyl) and undecylenic alcohol, and mixtures thereof, and more preferably from cetyl alcohol, stearyl alcohol, and cetearyl alcohol, PPG-5-Ceteth-10 phosphate, Oleth-3 phosphate, Oleth-10 phosphate, Ceteth-10 phosphate, a mixture of Ceteth-10 phosphate and Dicetyl phosphate, Dicetyl phosphate, Cetyl phosphate, Stearyl phosphate, laureth-7, laureth-9, trideceth-10, trideceth-12, C12-13 pareth-3, C12-13 pareth-23, C11-15 pareth-7, PPG-5 ceteth-20, PEG-55 Propylene Glycol Oleate, glycereth-26, decyl glucoside, cetearyl glucoside, decyl lauryl glucoside, stearyl glucoside, coco-glucoside, cocamide MIPA, and mixtures thereof.

The at least one nonionic surfactant is present in the hair treatment compositions of the present disclosure in an amount of from about 0.01% to about 15% by weight, preferably from about 0.1% to about 15% by weight, more preferably from about 0.5% to about 12% by weight, or more preferably from about 1% to about 10% by weight, relative to the total weight of the composition, including all ranges and sub ranges therebetween.

In a particular embodiment, the total amount of nonionic surfactants is about 1%, 1.5%, 1.6%, 1.7%, 1.8%, 1.9%, 2%, 2, 2.1%, 2.2%, 2.3%, 2.4%, 2.5%, 2.6%, 2.7%, 2.8%, 2.9%, 3.0%, 3.5%, 4%, 4.5%, 5%, 5.5%, 6%, 6.5%, 7%, 7.5%, 8%, 8.5%, 9%, 9.5%, or 10%, by weight, relative to the total weight of the composition.

### Thickening Agent

In some embodiments of the instant disclosure, the hair treatment compositions include at least one thickening agent (also referred to as thickeners or viscosity modifying agents). Non-limiting examples of thickening agents include xanthan gum, guar gum, biosaccharide gum, cellulose, acacia gum, Seneca gum, sclerotium gum, agarose, pectin, gellan gum, hyaluronic acid. Additionally, the one or more thickeners may include polymeric thickeners selected from ammonium polyacryloyldimethyl taurate, ammonium acryloyldimethyltaurate/VP copolymer, sodium polyacrylate, acrylates copolymers, polyacrylamide, carbomer, and acrylates/C10-30 alkyl acrylate crosspolymer. In some cases, the thickening agent includes ammonium polyacryloyldimethyl taurate and/or sodium polyacrylate.

Many thickening agents are water-soluble, and increase the viscosity of water or form an aqueous gel when the hair treatment composition of the disclosure is dispersed/dissolved in water. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water. Non-limiting examples of various types of thickeners include:

a. Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol 900 series from B.F. Goodrich (e.g., Carbopol® 954). In addition, other suitable carboxylic acid polymeric agents include Ultrez® 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as Carbopol 1342, Carbopol® 1382, Pemulen TR-1, and Pemulen TR-2, from B.F. Goodrich. In other words, examples of carboxylic acid polymer thickeners useful herein are those selected from carbomers, acrylates/C10-C30 alkyl acrylate crosspolymers, and mixtures thereof.

b. Crosslinked Polyacrylate Polymers

The hair treatment compositions of the instant disclosure can optionally contain crosslinked polyacrylate polymers useful as thickeners or gelling agents including both cationic and nonionic polymers. Examples of useful crosslinked nonionic polyacrylate polymers and crosslinked cationic polyacrylate polymers are those described in U.S. Pat. Nos. 5,100,660, 4,849,484, 4,835,206, 4,628,078 4,599,379 and EP 228,868, which are all incorporated herein by reference in their entirety.

c. Polyacrylamide Polymers

The hair treatment compositions of the instant disclosure can optionally contain polyacrylamide polymers, especially nonionic polyacrylamide polymers including substituted branched or unbranched polymers. Among these polyacrylamide polymers is the nonionic polymer given the CTFA designation polyacrylamide and isoparaffin and laureth-7, available under the Tradename Sepigel 305 from Seppic Corporation.

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc.

The hair treatment compositions may also contain thickening and texturising gels of the type as exemplified by the product range called Lubrajel® from United Guardian. These gels have moisturizing, viscosifying, stabilizing properties.

d. Polysaccharides

A wide variety of polysaccharides can be useful herein. “Polysaccharides” refer to gelling agents that contain a backbone of repeating sugar (i.e., carbohydrate) units. Nonlimiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation.

Other useful polysaccharides include scleroglucans comprising a linear chain of (1-3) linked glucose units with a (1-6) linked glucose every three units, a commercially available example of which is Clearogel™ CS11 from Michel Mercier Products Inc. In some instances, a mixture of sclerotium gum and xanthan gum is particularly useful.

e. Gums

Other thickening and gelling agents useful herein include materials which are primarily derived from natural sources. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof. In some instances, a mixture of sclerotium gum and xanthan gum is particularly useful.

Additional examples of water-soluble thickeners include water-soluble natural polymers, water-soluble synthetic polymers, clay minerals and silicic anhydride. Non-limiting examples of water-soluble natural polymers include gum arabic, tragacanth gum, karaya gum, guar gum, gellan gum, tara gum, locust bean gum, tamarind gum, sodium alginate, alginic acid propyleneglycol ester, carrageenan, farcelluran, agar, high-methoxy pectin, low-methoxy pectin, xanthine, chitosan, starch (for example starch derived from corn, potato, wheat, rice, sweet potato and tapioca, a-starch, soluble starch), fermentation polysaccharide (for example, xanthan gum, pullulan, carciran, dextran), acidic hetero-polysaccharide derived form callus of plants belonging to Polyantes sp. (for example, tuberous polysaccharide), proteins (for example, sodium casein, gelatin, albumin), chondroitin sulfate, and hyaluronic acid.

Non-limiting examples of water-soluble synthetic polymers include polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and starch derivatives (for example, starch oxide, dialdehyde starch, dextrin, British gum, acetyl starch, starch phosphate, carboxymethyl starch, hydroxyethyl starch, hydroxypropyl starch).

The at least one thickening agent may be present in the hair treatment compositions of the present disclosure in an amount of from about 0.01% to about 10% by weight, preferably from about 0.05% to about 8% by weight, more preferably from about 0.1% to about 8% by weight, or more preferably from about 0.1% to about 6%, evern more preferably from about 0.2% to about 5% by weight, relative to the total weight of the composition, including all ranges and sub ranges therebetween.

In a particular embodiment, the total amount of the thickening agent is about 0.01%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.1%, 4.2%, 4.3%, 4.4%, 4.5%, 4.6%, 4.7%, 4.8%, 4.9%, or 5%, by weight, relative to the total weight of the composition.

### Silicone Compound

The hair treatment composition according to the disclosure may also include one or more silicone compounds (also referred to as “silicones”), for example, dimethicone, amino silicones, dimethicone copolyols, etc.

The term “amino silicone” is intended to mean any silicone compound comprising at least one primary, secondary or tertiary amine or a quaternary ammonium group.

As amino silicone that may be used in the scope of the disclosure, the following can be cited:

a) polysiloxanes corresponding to formula (A):

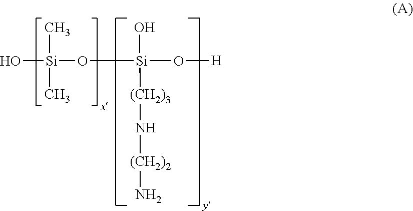

in which x′ and y′ are integers such that the weight-average molecular weight (Mw) is comprised between about 5000 and 500 000;

b) amino silicones corresponding to formula (B):

R′aG3-a-Si(OSiG2)n-(OSiGbR′2-b)m-O—SiG3-a-R′a (B)

in which:
G, which may be identical or different, designate a hydrogen atom, or a phenyl, OH or C1-C8 alkyl group, for example methyl, or C1-C8 alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula -CqH2qL in which q is a number ranging from 2 to 8 and L is an optionally quaternized amino group chosen from the following groups:

—NR″-Q-N(R″)2

—N(R″)2

—N+(R″)3A-

—N+H(R″)2A-

—N+H2(R″)A-

—N(R″)-Q-N+R″H2A-

—NR″-Q-N+(R″)2H A-

—NR″-Q-N+(R″)3A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, preferably from 2 to 4; and A- represents a cosmetically acceptable ion, in particular a halide such as fluoride, chloride, bromide or iodide.

A group of amino silicones corresponding to this definition (B) is represented by the silicones called “trimethylsilylamodimethicone” having formula (C):

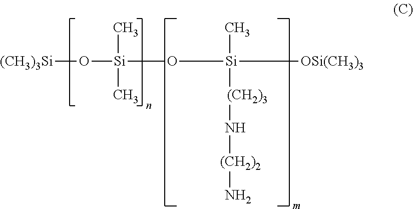

in which n and m have the meanings given above, in formula B.

Another group of amino silicones corresponding to this definition is represented by silicones having the following formulae (D) or (E):

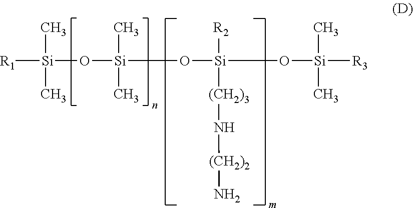

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R1, R2, R3, which may be identical or different, represent a hydroxy or C1-C4 alkoxy radical, where at least one of the radicals R1 to R3 denotes an alkoxy radical, preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

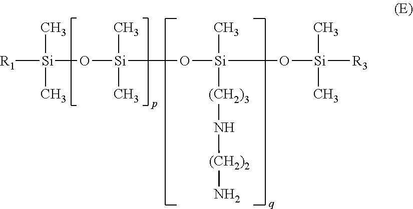

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R1, R2, which are different, represent a hydroxy or C1-C4 alkoxy radical, where at least one of the radicals R1 or R2 denotes an alkoxy radical, preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name Belsil® ADM 652.

A product containing amino silicones having structure (E) is sold by Wacker under the name Fluid WR 1300®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino silicones having formula (E), microemulsions are used whose average particle size ranges from 5 nm to 60 nanometres (limits included) and more preferably from 10 nm to 50 nanometres (limits included). Accordingly, according to the disclosure the microemulsions of amino silicone having formula (E) sold as Finish CT 96 E® or SLM 28020® by Wacker can be used.

Another group of amino silicones corresponding to this definition is represented by the following formula (F):

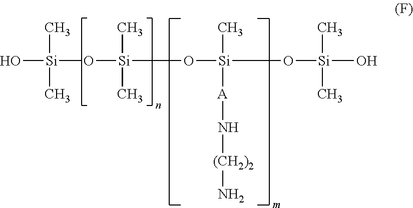

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino silicones corresponding to this definition is represented by the following formula (G):

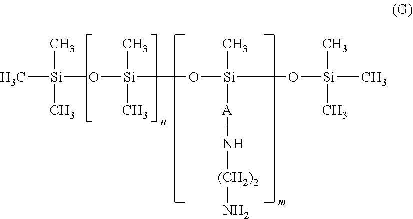

in which:
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

A silicone having this formula is for example DC2-8566 Amino Fluid by Dow Corning.

c) amino silicones corresponding to formula (H):

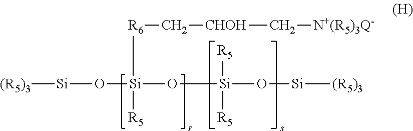

in which:
R5 represents a monovalent hydrocarbon-based radical containing from 1 to 18 carbon atoms, and in particular a C1-C18 alkyl or C2-C18 alkenyl radical, for example methyl;
R6 represents a divalent hydrocarbon-based radical, in particular a C1-C18 alkylene radical or a divalent C1-C18, for example C1-C8, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in U.S. Pat. No. 4,185,087.

d) quaternary ammonium silicones having formula (I):

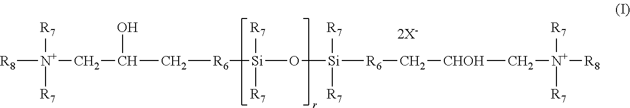

in which:
R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;
R6 represents a divalent hydrocarbon-based radical, in particular a C1-C18 alkylene radical or a divalent C1-C18, for example C1-C8, alkylenoxy radical linked to the Si via an SiC bond;
R8, which may be identical or different, represent a hydrogen atom, a monovalent hydrocarbon-based radical containing from 1 to 18 carbon atoms, and in particular a C1-C18 alkyl radical, a C2-C18 alkenyl radical or a —R6-NHCOR7 radical;
X— is an anion such as a halide ion, in particular chloride, or an organic acid salt (for example acetate);
r represents a mean statistical value from 2 to 200 and in particular from 5 to 100;

These silicones are described, for example, in patent application EP-A 0 530 974.

e) amino silicones having formula (J):

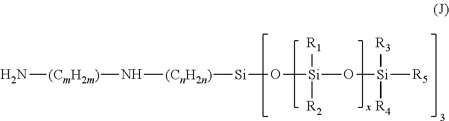

in which:
R1, R2, R3 and R4, which may be identical or different, denote a C1-C4 alkyl radical or a phenyl group;
R5 denotes a C1-C4 alkyl radical or a hydroxyl group;
n is an integer ranging from 1 to 5;
m is an integer ranging from 1 to 5;

and in which x is chosen such that the amine number is between 0.01 and 1 meq/g;

f) multiblock polyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe2O)xSiMe2-R—N(R″)—R′—O(C2H4O)a(C3H6O)b-R′—N(H)—R—]

or alternatively

[—(SiMe2O)xSiMe2-R—N(R″)—R′—O(C2H4O)a(C3H6O)b-]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C2-C12 hydrocarbon-based radical, optionally including one or more heteroatoms such as oxygen; preferably, R denotes an ethylene radical, a linear or branched propylene radical, a linear or branched butylene radical, or a —CH2CH2CH2OCH(OH)CH2- radical; preferentially R denotes a CH2CH2CH2OCH(OH)CH2- radical;
R′, which may be identical or different, represent a divalent linear or branched C2-C12 hydrocarbon-based radical, optionally including one or more heteroatoms such as oxygen; preferably, R′ denotes an ethylene radical, a linear or branched propylene radical, a linear or branched butylene radical, or a —CH2CH2CH2OCH(OH)CH2- radical; preferentially R′ denotes —CH(CH3)-CH2-.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names Silsoft™ A-843 or Silsoft™ A+ by Momentive.

g) the alkylamino silicones corresponding to formula (K) below:

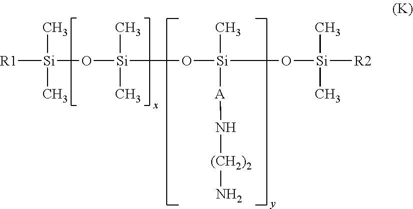

in which:
x and y are numbers ranging from 1 to 5000; preferably, x ranges from 10 to 2000 and especially from 100 to 1000; preferably, y ranges from 1 to 100;
R1 and R2, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms,

Preferably, A comprises 3 to 6 carbon atoms, especially 4 carbon atoms; preferably, A is branched. Mention may be made especially of the following divalent radicals: —CH2CH2CH2 and —CH2CH(CH3)CH2-.

Preferably, R1 and R2, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:
x ranging from 10 to 2000 and especially from 100 to 1000;
y ranging from 1 to 100;
A comprising 3 to 6 carbon atoms and especially 4 carbon atoms; preferably, A is branched; and more particularly A is chosen from the following divalent radicals: —CH2CH2CH2 and —CH2CH(CH3)CH2-; and
R1 and R2, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A preferred silicone of formula (K) is bis-cetearyl amodimethicone (INCI name). Mention may be made especially of the silicone sold under the name Silsoft™ AX by Momentive.

In one embodiment, the amino silicones according to the disclosure are chosen from amodimethicone, trideceth-9 PG amodimethicone, PEG-40/PPG-8 Methylaminopropyl/hydroxypropyl dimethicone copolymer, and mixtures thereof.

Preferably, the amino silicones according to the disclosure are chosen from the amino silicones of formula (F). A preferred silicone of formula (F) is amodimethicone (INCI name) sold under the tradename XIAMETER® MEM-8299 Cationic Emulsion by Dow Corning.

The silicones other than the amino silicones above can be chosen from dimethicone and dimethicone copolyol compounds such as oxypropylenated and/or oxyethylenated polydimethyl(methyl)siloxane, oxypropylenated and/or oxyethylenated polymethyl (C8-C22) alkyl dimethyl methyl siloxane, and mixtures thereof, and wherein the dimethicone copolyol compounds are preferably selected from Dimethicone PEG-8 Benzoate, Dimethicone PEG-7 Phosphate, Dimethicone PEG-8 Phosphate, Dimethicone PEG-10 Phosphate, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, and mixtures thereof.

In some embodiments, the at least one silicone compound in the hair treatment compositions of the present disclosure include dimethicone and/or amodimethicone.

In one embodiment, the at least one silicone compound in the hair treatment compositions of the present disclosure comprise dimethicone and amodimethicone.

The at least one silicone compound in the hair treatment composition of the present disclosure may be present in an amount of from about 0.1% to about 15% by weight, preferably from about 0.5% to about 10% by weight, and most preferably from about 1% to about 5% by weight, relative to the total weight of the composition, including all ranges and sub ranges therebetween.

In a particular embodiment, the total amount of the silicone compound(s) is about 0.1% 0.02%, 0.3%, 0.4%, 0.5%, 0.6%, 0.7%, 0.8%, 0.9%, 1%, 1.5%, 2%, 2.5%, 3%, 3.5%, 4%, 4.1%, 4.2%, 4.3%, 4.4%, 4.5%, 4.6%, 4.7%, 4.8%, 4.9%, or 5%, by weight, relative to the total weight of the composition.

### pH

The pH of the hair treatment compositions of the present disclosure may range from about 2 to less than 7, or from about pH 2 to about 6.5, or from about pH 2.1 to about 6, or from about pH 2.2 to about 5.2, such as from about pH 2.2 to about 5, or preferably from about pH 2.2 to about 4.8, or more preferably from about pH 2.2 to about 4.5, or even more preferably from about pH 2.2 to about 4, including all ranges and sub ranges therebetween.

In some embodiments, the pH of the hair treatment compositions of the present disclosure can be about 2, 2.1, 2.2, 2.3, 2.4, 2.5, 2.6, 2.75, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5.

The desired pH may be obtained by using one or more of the alkaline agents described above.

The desired pH may be obtained by using one or more of the alkaline agents described above.

### Water

The compositions according to the disclosure include water. The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The composition according to the disclosure comprises water in an amount ranging from about 50% to about 99% by weight, preferably from about 65% to about 95% by weight, and most preferably from about 70% to about 92% by weight, relative to the total weight of the composition.

### Water-Soluble Solvent

According to at least one embodiment, the hair treatment compositions of the instant disclosure include at least one water-soluble solvent. The term “water-soluble solvent” is interchangeable with the term “water-miscible solvent” and means a compound that at 25° C. and at atmospheric pressure (760 mmHg) has a solubility of at least 50% in water. In some cases, the water-soluble solvent has a solubility of at least 60%, 70%, 80%, or 90% in water at 25° C. and at atmospheric pressure (760 mmHg). Non-limiting examples of water-soluble solvents include, for example, glycerin, alcohols (for example, C₁₋₈ or C₁₋₄ alcohols), organic solvents, polyols, glycols, and a mixture thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylne glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some embodiments, the water-soluble solvent may be selected from one or more glycols, C₁₋₄ alcohols, glycerin, and a mixture thereof.

The total amount of the at least one water-soluble solvent in the hair treatment composition may vary but is typically about 0.01% to 10% by weight, preferably in an amount of from about 0.05% to 8% by weight, more preferably from about 0.1 to about 5% by weight, based on the total weight of the composition.

In various embodiments, the hair treatment composition of the present disclosure is a leave-on conditioner composition in the form of an aqueous composition or an emulsion or a cream or a lotion.

In one embodiment, the hair treatment compositions of the present disclosure is a leave-on composition for conditioning and/or for keeping hair in a particular configuration or shape (such as a styling composition).

In one embodiment, the present disclosure relates to a method or process of treating hair, wherein the hair treatment composition is applied onto hair (wet or dry), massaged into the hair fibers, then allowed to remain on the hair for at least one hour.

In one embodiment, the present disclosure relates to a process or method of treating or conditioning hair according to the general protocol or treatment cycle comprising:

a) optionally, washing the hair with a shampoo and/or rinsing the hair with water, and optionally drying the hair at a temperature ranging from room temperature to about 200° C. (for example, with blow drying) and optionally applying a smoothing action on the hair;

b) applying a hair treatment composition according to the instant disclosure onto the hair and allowing the hair treatment composition to remain on the hair for a period of time ranging from about 1 minute to about 1 hour, from about 15 minutes to about 45 minutes, or for about 30 minutes;

c) after allowing the hair treatment composition to remain on the hair for a period of time, optionally rinsing the hair treatment composition from the hair (preferably, the hair treatment composition is rinsed from the hair but rinsing it from the hair is not required);

d) drying the hair at a temperature ranging from room temperature up to about 200° C., for example, using a blow dryer, while optionally applying a smoothing action on the hair;

e) passing a flat iron over the hair, for example, passing the flat iron over the hair at least once (or at least 2 times, at least 4 times, at least 5 times, or at least 10 times or more, for example, up to 20, 22, 25, 28, 30, 40, 50, 75, or 100 times);

f) after passing the flat iron over the hair, optionally applying a neutralizing composition to the hair (for example, a neutralizing composition comprising hydrogen peroxide) and allowing the neutralizing composition to remain on the hair for a period of time ranging from about 1 minute to about 1 hour; about 5 minutes to about 30 minutes, or about 10 minutes;

g) washing the hair with a shampoo and/or a conditioner and/or rinsing the hair with water; and

h) drying the hair at a temperature ranging from room temperature up to about 200° C., for example, using a blow dryer, while optionally applying a smoothing action on the hair.

The smoothing action may be conducted by brushing or combing or passing the fingers through the hair.

Drying the hair at a temperature ranging from room temperature up to about 200° C. may be accomplished by drying the hair with blow dryer device or using other heat sources such as a flat iron, a hair dryer, a heat lamp, a heat wand, or other similar devices.

When the method above comprises step e), the flat iron may be employed at a temperature of about 100° C., or ranging from about 100° C. to about 250° C. or from about 110° C. to about 230° C. or from about 110° C. to about 210° C. or from about 120° C. to about 200° C. or from about 150° C. to about 190° C., or from about 190° C. to about 250° C., including ranges and sub-ranges there between, or at a temperature of about 230° C. or about 225° C. or about 220° C. or about 210° C. or about 200° C. or about 190° C. or about 180° C. or about 150° C. or about 100° C. and preferably at about 230° C. or at about 230° C.

In various embodiments, the flat iron is passed over the hair at least 1, 2, 3, 4, 5, 6, 7, 8, 9, 10 times or more, for example, up to 20, 22, 25, 28, or 30 times.

In an embodiment, after passing the flat iron over the hair, the above-described method includes a step of applying a neutralizing composition to the hair (for example, a neutralizing composition comprising an oxidizing agent such as hydrogen peroxide) and allowing the neutralizing composition to remain on the hair for a period of time ranging from about 1 minute to about 1 hour; about 5 minutes to about 30 minutes, or about 10 minutes.

The hair treatment composition can be applied onto the hair using an applicator device or with the hands or gloved hands or with the fingers. A suitable applicator device is an applicator brush or applicator comb or applicator spatula or a dispenser or applicator tip attached to the container holding the composition.

The method or treatment cycle described above may be repeated over a period of days or weeks.

The method/process in accordance with the present disclosure may be followed by or be preceded by the use of rinse-off compositions for cleansing and/or conditioning hair, such as a shampoo or conditioner.

The cosmetic effects imparted by the hair treatment compositions and accompanying methods of treating the hair of the present disclosure may be evaluated by visually assessing the appearance of the hair after processing the hair according to the methods of the disclosure. Another type of evaluation can also involve sensorial evaluations of the hair.

It was surprisingly and unexpectedly discovered that the hair contacted with the hair treatment compositions of the present disclosure and treated according to the methods of the disclosure visually generally appeared to be less volumized, and less frizzy compared to hair contacted with hair treatment compositions that did not contain thiolactic acid. Using the protocol with heat (step e), the hair appeared to be more extended and/or straight, and without this step, the curls were much more defined. It was also surprisingly and unexpectedly discovered that the hair contacted with the hair treatment compositions of the disclosure and treated according to the methods of the disclosure were smoother to the touch, more manageable, more disciplined (i.e., less or no fly-aways), and exhibited more regularity with respect to shape and appearance. These effects were even more observable after subjecting the hair to multiple treatment or application cycles.

The hair treatment compositions of the present disclosure may be packaged in any suitable container such as a tube, a jar or a bottle. In certain embodiments, the composition can be packaged in a tube or bottle, for example, a squeeze tube or squeeze bottle. Additionally, an applicator device can be attached or connected to the opening of the packaging/squeeze tube or bottle wherein the applicator device is a brush or a comb with teeth such that the ends of the teeth have openings from which the composition of the disclosure can flow through and be applied directly onto the hair.

The composition of the present disclosure may also be provided as component of a kit for treating hair wherein the kit can additionally contain other components such as a shampoo and/or a conditioner and/or hair masque and/or ampoule.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The hair treatment compositions according to the disclosure can be manufactured by known processes used generally in the cosmetics.

The following Examples are intended to be non-restrictive and explanatory only, with the scope of the disclosed embodiments.

The ingredient amounts in the hair treatment compositions/formulas described below are expressed in % by weight, based on the total weight of the composition.

### EXAMPLES

### Example A

### Formulations

The formulations of the present disclosure can be prepared in water according to Table 1:

TABLE 1

| | % by Weight |
|---|---|
| Ingredient | (active matter, “AM”) |
| Thiolactic acid | 0.1-20 |
| Non-Polymeric and Non-Thiol, Mono-, Di-, | 0.1-20 |
| and/or Tricarboxylic acid (e.g., glutaric acid, | |
| citric acid, lactic acid, malic acid, etc.) | |
| Alkaline agent (e.g., alkanolamine | 0-15 |
| such as triethanolamine, monoethanolamine, | |
| aminomethyl propanol, etc.) | |
| Cationic surfactants | 0.1-15 |
| (e.g., cetrimonium chloride, behentrimonium | |
| chloride, | |
| dipalmitoylethylhydroxyethylmethylammonium | |
| methosulfate, behentrimonium methosulfate, | |
| and mixtures thereof) | |
| Nonionic Surfactant (e.g., fatty alcohol and | 0.1-15 |
| alkoxylated fatty alcohols, etc.) | |
| Thickening Agent (e.g., thickening gums, etc.) | 0.01-10 |
| Silicone Compounds (e.g., dimethicone, | 0.1-15 |
| aminosilicones such as amodimethicone, | |
| dimethicone copolyols, etc.) | |
| Water-Soluble Solvent (e.g., glycols, C1-4 | 0.01-15 |
| alcohols, glycerin, etc.) | |
| Water, organic solvent, and additional | Q.S. 100 |
| components such as fragrance, preservatives, | |
| pH adjusting agents, plant extracts, plant oils, | |
| hydrolyzed proteins, vitamins, salts, fragrances, | |
| opacifiers (e.g., glycol distearate), plasticizers, | |
| salts, vitamins, sunscreens, lower alkanes, | |
| hydrocarbons, colorants, or mixtures thereof | |
| (as needed or desired) | |
| pH | 2 to 7 |

Comparative formulations and formulations according to Table 1 were prepared and are presented below in TABLE 2.

TABLE 2
Hair Formulations

| | | Comp. | Comp. | Comp. | | | | | | | | | |
|---|---|---|---|---|---|---|---|---|---|---|---|---|---|
| | | 1 | 2 | 3 | 1 | 2 | 3 | 4 | 5 | 6 | 7 | 8 | 9 |
| | INCI US | wt. % | wt % | wt. % | wt. % | wt. % | wt. % | wt. % | wt. % | wt. % | wt. % | wt. % | wt. % |
| TLA | THIOLACTIC ACID | 8 | 8 | 8 | 8 | 8 | 8 | 8 | 8 | 8 | 8 | 8 | 8 |
| Mono-carboxylic | LACTIC ACID | | | | | 7.2 | | | 7.2 | 1.8 | | | |
| Acid | MALIC ACID | | | | | | | 10 | 2 | | 4 | | |
| Dicarboxylic Acid | GLUTARIC ACID | | | | | | 10 | | | 2 | 4 | | |
| Tricarboxylic Acid | CITRIC ACID | | | | 6 | | | | | | | 5 | 5 |
| Alkaline Agent | TRIETHANOLAMINE | | 0.3 | 3 | 2.3 | 0.7 | 0.5 | 1.1 | 1.3 | 0.3 | 0.5 | 1 | 6 |
| Cationic | BEHENTRIMONIUM | 3.2 | 3.2 | 3.2 | 3.2 | 3.2 | 3.2 | 3.2 | 3.2 | 3.2 | 3.2 | 3.2 | 3.2 |
| Surfactant | CHLORIDE | | | | | | | | | | | | |
| Nonionic | CETEARYL ALCOHOL | 6 | 6 | 6 | 6 | 6 | 6 | 6 | 6 | 6 | 6 | 6 | 6 |
| Surfactant | AND/OR STEARETH-20 | | | | | | | | | | | | |
| Thickening Agent | XANTHAN GUM | 0.8 | 0.8 | 0.8 | 0.8 | 0.8 | 0.8 | 0.8 | 0.8 | 0.8 | 0.8 | 0.8 | 0.8 |
| | AND/OR SCLEROTIUM | | | | | | | | | | | | |
| | GUM | | | | | | | | | | | | |
| Silicone | DIMETHICONE AND/OR | 2 | 2 | 2 | 2 | 2 | 2 | 2 | 2 | 2 | 2 | 2 | 2 |
| | AMODIMETHICONE | | | | | | | | | | | | |
| Water- | ISOPROPYL ALCOHOL | 0.7 | 0.7 | 0.7 | 0.7 | 0.7 | 0.7 | 0.7 | 0.7 | 0.7 | 0.7 | 0.7 | 0.7 |
| Soluble Solvent | | | | | | | | | | | | | |
| Misc. | PRESERVATIVE AND | ≤2 | ≤2 | ≤2 | ≤2 | ≤2 | ≤2 | ≤2 | ≤2 | ≤2 | ≤2 | ≤2 | ≤2 |
| | FRAGRANCE | | | | | | | | | | | | |
| Water | WATER | Q.S. | Q.S. | Q.S. | Q.S. | Q.S. | Q.S. | Q.S. | Q.S. | Q.S. | Q.S. | Q.S. | Q.S. |
| | | 100 | 100 | 100 | 100 | 100 | 100 | 100 | 100 | 100 | 100 | 100 | 100 |

| | | | | | | | | | | | | |
|---|---|---|---|---|---|---|---|---|---|---|---|---|
| Total mono-, di-, and/or tricarboxylic acid | | | | 6 | 7.2 | 10.0 | 10.0 | 9.2 | 2.8 | 8.0 | 5 | 5 |
| Total Alkaline Agent | | | | 2.3 | 0.7 | 0.5 | 1.1 | 1.3 | 0.3 | 0.5 | 1 | 6 |
| pH | 3.5 | 2.2 | 3.5 | 2.8 | 2.2 | 2.2 | 2.2 | 2.2 | 2.2 | 2.2 | 2.2 | 3.5 |

### Example B

### Performance Evaluation

The formulas above were used to treat hair swatches as follows:

1. The hair swatches were washed with a shampoo having a neutral pH, rinsed with water, and the hair was dried with a blow dryer at a temperature ranging from room temperature up to about 100° C. or up to about 160° C., while applying a smoothing action on the hair.

2. Each formulation was spread along the length of the hair swatches and allowed to remain on the hair for about 30 minutes before rinsed from the hair.

3. After rinsing the formulations from the hair, the hair was dried with a blow dryer at a temperature of up to about 100° C. or up to about 160° C., while applying a smoothing action on the hair.

4. A flat iron at a temperature of about 230° C. was passed over each hair swatch 10 times.

5. The hair swatches were then treated with a neutralizing cream comprising hydrogen peroxide, which was allowed to remain on the hair for about 10 minutes before being rinsed from the hair;

6. The hair swatches were then washed with a shampoo having a neutral pH, rinsed with water, and allowed to air dry.

The hair swatches were assessed visually with respect to the attributes of straightening, volume (i.e., volume control or reduction) and frizz (i.e., frizz control or reduction) based on a standardized rating scale of 1 to 4, with 4 being the best rating (highest degree of straightening, least amount of volume and least amount of frizziness). The swatches were tested immediately after step (6) and again after being shampooed an additional ten times. The results are reported below.

Visual Assesment

| | None | Comp 1 | Comp 2 | Comp 3 | 2 | 3 | 4 | 5 | 6 | 7 | 8 | 9 |
|---|---|---|---|---|---|---|---|---|---|---|---|---|
| Initial | 1 | 1.5 | 1.5 | 1.5 | 2.2 | 1.5 | 1.7 | 1.5 | 1.8 | 2.5 | 2.5 | 2.5 |
| After 10X | 1 | 1.5 | 1.5 | 1.5 | 1.8 | 1.7 | 2.0 | 1.8 | 1.7 | 2.4 | NA | NA |
| Shampoo | | | | | | | | | | | | |

NA = Not Application (was not tested)

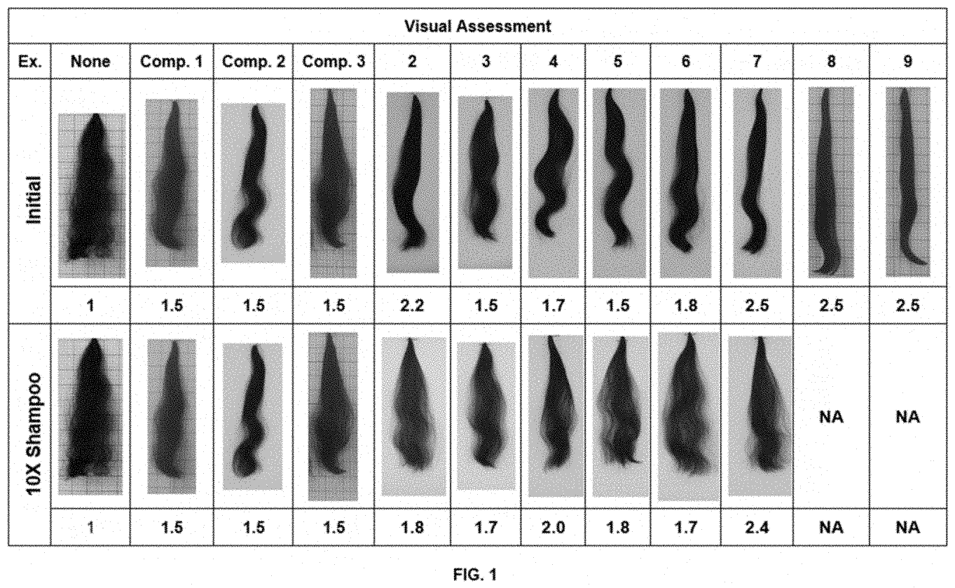

Pictures of the hair swatches and the visual assessment scores are also shown in FIG. 1. The data show that hair swatches treated with the inventive compositions were straighter, had better volume control, and were less frizzy than both untreated hair swatches and hair swatches treated with the comparative compositions in Table 2. The data also show long lasting effects such as volume control and less frizziness even after 10 shampooings.

1. A hair treatment composition comprising:
at least one reducing agent selected from thiol-based compounds, non-thiol-based compounds, and a mixture thereof;
at least one non-polymeric and non-thiol, mono-, di-, and/or tri-carboxylic acid, and/or a salt thereof, or a mixture thereof;
at least one alkaline agent;
optionally, at least one cationic surfactant; and
water,
wherein the pH of the composition is from about 2 to less than 7.

2. The hair treatment composition of claim 1, wherein the at least one reducing agent is a thiol-based compound selected from thiolactic acid and derivatives thereof, 3-mercaptopropionic acid and derivatives thereof, cysteamine and derivatives thereof, cysteine and derivatives thereof, thioglycolic acid and derivatives thereof, and mixtures thereof.

3. The hair treatment composition of claim 1, wherein the at least one reducing agent is a thiol-based compound selected from thiolactic acid, glycerol monothiolactate, ammonium thiolactate, MEA-thiolactate, 3-mercaptopropionic acid, glycerol 3-mercaptopropionate, ethyleneglycol 3-mercaptopropionate, cysteamine, N-acetylcysteamine, N-propionylcysteamine, cysteine, N-acetylcysteine, N-alkanoylcysteine, cysteine alkyl esters, homocysteine, thioglycolic acid, ethanolamine thioglycolate, glyceryl thioglycolate, glutathione, thioglycerol, thiomalic acid, thiodiglycol, 2-mercaptoethanol, dithiothreitol, thioxanthine, thiosalicylic acid, thiopropionic acid, lipoic acid, and mixtures thereof.

4. The hair treatment composition of claim 1, wherein the at least one reducing agent comprises thiolactic acid.

5. The hair treatment composition of claim 1 claims comprising:
about 0.1 to about 20 wt % of the at least one reducing agent, based on the total weight of the hair treatment composition.

6. The hair treatment composition of claim 1, wherein the at least one non-polymeric and non-thiol, mono-, di-, and/or tri-carboxylic acid comprises:
at least one monocarboxylic acid selected from formic acid, acetic acid, lactic acid, propionic acid, butyric acid, gluconic acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, and arachidic acid, a salt thereof, and a mixture thereof;
at least one dicarboxylic acid selected from oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid, a salt thereof, and a mixture thereof; and/or
at least one tricarboxylic acid selected from citric acid, isocitric acid, aconitric acid, propane-1,2,3-tricarboxylic acid, and benzene-1,3,5-tricarboxylic acid, a salt thereof, and a mixture thereof.

7. The hair treatment composition of claim 1 comprising:
about 0.1 to about 20 wt. % of the at least one non-polymeric and non-thiol, mono-, di-, and/or tri-carboxylic acid, a salt thereof, or a mixture thereof, based on the total weight of the hair treatment composition.

8. The hair treatment composition of claim 1, wherein the at least one alkaline agent is selected from organic amines, an amino acid, an ammonium salt, an alkali metal hydroxide, an alkali earth metal hydroxide, an alkali metal carbonate, an alkali metal phosphate, an alkali metal citrate, and mixtures thereof.

9. The hair treatment composition of claim 1, wherein the at least one alkaline agent comprises an organic amine selected from monoethanolamine, diethanolamine, triethanolamine, aminomethyl propanol, tromethamine, monoisopropanolamine, diisopropanolamine, triisopropanolamine, and a mixture thereof, preferably triethanolamine.

10. (canceled)

11. The hair treatment composition of claim 9, wherein the organic amine is a diamine selected from tetrahydroxyethyl ethylenediamine, tetrahydroxypropyl ethylenediamine, tetrahydroxypropyl ethylenediamine dioleate, 2,2′-(ethylenedioxy)bis(ethylamine), 4,7,10-trioxa-1,13-tridecanediamine, and a mixture thereof.

12. The hair treatment composition of claim 1 comprising:
about 0.01 to about 15 wt. % of the at least one alkaline agent, based on the total weight of the hair treatment composition.

13. (canceled)

14. The hair treatment composition of claim 1 comprising:
about 0.1 to about 15 wt. % of the at least one cationic surfactant selected from primary, secondary, tertiary fatty amine salts, quaternary ammonium compounds and their salts, and mixtures thereof, based on the total weight of the hair treatment composition.

15. The hair treatment composition of claim 1, further comprising: about 0.1 to about 15 wt. % of at least one nonionic surfactant selected from fatty alcohols, alkoxylated fatty alcohols, alkyl(ether)phosphates, alkylpolyglucosides, fatty acid alkanolamides, and mixtures thereof, based on the total weight of the hair treatment composition.

16. (canceled)

17. (canceled)

18. The hair treatment composition of claim 1 further comprising:
about 0.01 to about 10 wt. % at least one thickening agent selected, based on the total weight of the hair treatment composition, wherein the at least one thickening agent is selected from cellulose polymers, gums, modified or unmodified carboxyvinyl polymers, polyacrylamides, copolymers of acrylic acid and of acrylamide, sodium salts of polyhydroxycarboxylic acids, 2-acrylamido-2-methylpropanesulphonic acid polymers and copolymers, polyacrylic acid/alkyl acrylate, glucans, modified or unmodified starches, silicas, and mixtures thereof.

19. (canceled)

20. (canceled)

21. (canceled)

22. The hair treatment composition of claim 1 further comprising:
about 0.1 to about 15 wt. % of at least one silicone compound based on the total weight of the hair treatment composition, wherein the at least one silicone is selected from dimethicone; aminosilicones; dimethicone copolyol selected from oxypropylenated and/or oxyethylenated polydimethyl(methyl)siloxane, oxypropylenated and/or oxyethylenated polymethyl (C8-C22) alkyl dimethyl methyl siloxane, and mixtures thereof.

23. (canceled)

24. (canceled)

25. A hair treatment composition comprising:
about 0.1 to about 20 wt. % of at least one thiol-based compound selected from thiolactic acid, esters of thiolactic acid, salts thereof, and mixtures thereof;
about 0.1 to about 20 wt. % of at least one non-polymeric and non-thiol, mono-, di-, and/or tri-carboxylic acid;
about 0.01 to about 15 wt. % of the at least one alkaline agent selected from organic amines, alkali metal hydroxides, alkali earth metal hydroxides, alkali metal carbonates, alkali metal phosphates, alkali metal citrates, and mixtures thereof;
about 0.1 to about 15 wt. % of at least one cationic surfactant selected from primary, secondary, tertiary fatty amine salts, quaternary ammonium compounds and their salts, and mixtures thereof;
about 0.1 to about 15 wt. % of at least one nonionic surfactant selected from fatty alcohols, alkoxylated fatty alcohols, alkyl(ether)phosphates, alkylpolyglucosides, fatty acid alkanolamides, and mixtures thereof;
about 0.01 to about 10 of the at least one thickening agent selected from cellulose polymers, gums, modified or unmodified carboxyvinyl polymers, polyacrylamides, copolymers of acrylic acid and of acrylamide, sodium salts of polyhydroxycarboxylic acids, 2 acrylamido-2-methylpropanesulphonic acid polymers and copolymers, polyacrylic acid/alkyl acrylate, glucans, modified or unmodified starches, silicas, and mixtures thereof;
about 0.1 to about 15 wt. % of the least one silicone compound selected from dimethicone; aminosilicones; dimethicone copolyol selected from oxypropylenated and/or oxyethylenated polydimethyl(methyl)siloxane, oxypropylenated and/or oxyethylenated polymethyl (C8-C22) alkyl dimethyl methyl siloxane, and mixtures thereof; and
water, wherein the pH of the composition is less than 7.

26. The hair treatment composition of claim 25 comprising:
about 0.1 to about 20 wt. % of thiolactic acid;
about 0.1 to about 20 wt. % of at least one non-polymeric non thiol, mono-, di-, and/or tri-carboxylic acid;
about 0.01 to about 15 wt. % of the at least one alkaline agent, wherein the at least one alkaline agent is selected from monoethanolamine, diethanolamine, triethanolamine, aminomethyl propanol, tris(hydroxymethyl)aminomethane, tetra hydroxy propyl ethylenediamine, 2,2′-(ethylenedioxy)bis(ethylamine), 4,7,10-trioxa-1,13-tridecanediamine, and a mixture thereof;
about 0.1 to about 15 wt. % of at least one cationic surfactant, wherein the at least one cationic surfactant is a quaternary ammonium compound;
about 0.1 to about 15 wt. % of at least one nonionic surfactant selected from fatty alcohols, alkoxylated fatty alcohols, alkyl(ether)phosphates, alkylpolyglucosides, fatty acid alkanolamides, and mixtures thereof;
about 0.01 to about 10 of the at least one thickening agent, wherein the at least one thickening agent is a gum; and
about 0.1 to about 15 wt. % of the least one silicone compound selected from dimethicone, amodimethicone, and a mixture thereof; and
water.

27. A method of treating hair, the method comprising:
(a) optionally, washing/rinsing the hair with a shampoo and/or rinsing the hair with water, and optionally drying the hair at a temperature ranging from room temperature to about 200° C.;
(b) applying the hair treatment composition of claim 1 onto the hair;
(c) allowing the hair treatment composition in (b) to remain on the hair for a period of time ranging from about 1 minute to about 1 hour, and optionally, rinsing the hair treatment composition from the hair;
(d) drying the hair at a temperature ranging from room temperature up to about 200° C., while optionally applying a smoothing action on the hair;
(e) optionally, passing a flat iron over the hair swatch at least once; and
optionally, allowing the hair treatment composition to remain on the hair for at least 1 hour or for a period of time ranging from 1 hour up to 72 hours;
(g) optionally, applying a neutralizing composition to the hair and allowing the neutralizing composition to remain on the hair for a period of time; and
(h) washing/rinsing the hair with a shampoo and/or rinsing the hair with water, followed by allowing the hair to air dry, while optionally applying a smoothing action on the hair.

28. The method of the preceding claim 27, wherein the flat iron is employed at a temperature ranging from 100° C. to 250° C.

29. The method of claim 27, wherein step (e) includes passing the flat iron over the hair at least 3 times.

30. (canceled)

31. (canceled)

32. (canceled)
